SEQUENCE LISTING (44 sequences)

SEQ ID NO: 1 - protein, 117 aa, Homo sapiens
PLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 408 aa, Homo sapiens
MTSKGPEEEHPSVTLFRQYLRIRTVQPKPDYGAAVAFFEETARQLGLGCQKVEVAPGYVVTVLTWPGTNPTLSSILLNSHTDVVPVFKEHWSHDPFEAFKDSEGYIYARGAQDMKCVSIQYLEAVRRLKVEGHRFPRTIHMTFVPDEEVGGHQGMELFVQRPEFHALRAGFALDEGIANPTDAFTVFYSERSPWWVRVTSTGRPGHASRFMEDTAAEKLHKVVNSILAFREKEWQRLQSNPHLKEGSVTSVNLTKLEGGVAYNVIPATMSASFDFRVAPDVDFKAFEEQLQSWCQAAGEGVTLEFAQKWMHPQVTPTDDSNPWWAAFSRVCKDMNLTLEPEIMPAATDNRYIRAVGVPALGFSPMNRTPVLLHDHDERLHEAVFLRGVDIYTRLLPALASVPALPSDS

SEQ ID NO: 3 - protein, 324 aa, Homo sapiens
AASCVLLHTGQKMPLIGLGTWKSEPGQVKAAVKYALSVGYRHIDCAAIYGNEPEIGEALKEDVGPGKAVPREELFVTSKLWNTKHHPEDVEPALRKTLADLQLEYLDLYLMHWPYAFERGDNPFPKNADGTICYDSTHYKETWKALEALVAKGLVQALGLSNFNSRQIDDILSVASVRPAVLQVECHPYLAQNELIAHCQARGLEVTAYSPLGSSDRAWRDPDEPVLLEEPVVLALAEKYGRSPAQILLRWQVQRKVICIPKSITPSRILQNIKVFDFTFSPEEMKQLNALNKNWRYIVPMLTVDGKRVPRDAGHPLYPFNDPY

SEQ ID NO: 4 - protein, 364 aa, Homo sapiens
MAHRFPALTQEQKKELSEIAQSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAANKEATQEAFMKRAMANCQAAKGQYVHTGSSGAASTQSLFTACYTY

SEQ ID NO: 5 - protein, 319 aa, Homo sapiens
MATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 6 - protein, 296 aa, Homo sapiens
MNTEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 7 - protein, 206 aa, Homo sapiens
MAVKKIAIFGATGQTGLTTLAQAVQAGYEVTVLVRDSSRLPSEGPRPAHVVVGDVLQAADVDKTVAGQDAVIVLLGTRNDLSPTTVMSEGARNIVAAMKAHGVDKVVACTSAFLLWDPTKVPPRLQAVTDDHIRMHKVLRESGLKYVAVMPPHIGDQPLTGAYTVTLDGRGPSRVISKHDLGHFMLRCLTTDEYDGHSTYPSHQYQ

SEQ ID NO: 8 - protein, 241 aa, Homo sapiens
GGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAA

SEQ ID NO: 9 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 10 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 11 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 12 - protein, 475 aa, Homo sapiens [sequence omitted]

SEQ ID NO: 13 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 14 - protein, 434 aa, Homo sapiens
MSILKIHAREIFDSRGNPTVEVDLFTSKGLFRAAVPSGASTGIYEALELRDNDKARYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAGVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTAIAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 15 - protein, 143 aa, Homo sapiens
SLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 16 - protein, 445 aa, Homo sapiens
MNEEYDVIVLGTGLTECILSGIMSVNGKKVLHMDRNPYYGGESASITPLEDLYKRFKIPGSPPESMGRGRDWNVDLIPKFLMANGQLVKMLLYTEVTRYLDFKVTEGSFVYKGGKIYKVPSTEAEALASSLMGLFEKRRFRKFLVYVANFDEKDPRTFEGIDPKKTTMRDVYKKFDLGQDVIDFTGHALALYRTDDYLDQPCYETINRIKLYSESLARYGKSPYLYPLYGLGELPQGFARLSAIYGGTYMLNKPIEEIIVQNGKVIGVKSEGEIARCKQLICDPSYVKDRVEKVGQVIRVICILSHPIKNTNDANSCQIIIPQNQVNRKSDIYVCMISFAHNVAAQGKYIAIVSTTVETKEPEKEIRPALELLEPIEQKFVSISDLLVPKDLGTESQIFISRTYDATTHFETTCDDIKNIYKRMTGSEFDFEEMKRKKNDIYGED

SEQ ID NO: 17 - protein, 145 aa, Homo sapiens
LNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 18 - protein, 474 aa, Homo sapiens
MATNWGSLLQDKQQLEELARQAVDRALAEGVLLRTSQEPTSSEVVSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV

SEQ ID NO: 19 - protein, 208 aa, Homo sapiens
PPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPMDEKSL

SEQ ID NO: 20 - protein, 414 aa, Homo sapiens [sequence omitted]

SEQ ID NO: 21 - protein, 356 aa, Homo sapiens
MAAEGWIWRWGWGRRCLGRPGLLGPGPGPTTPLFLLLLLGSVTADITDGNSEHLKREHSLIKPYQGVGSSSMPLWDFQGSTMLTSQYVRLTPDERSKEGSIWNHQPCFLKDWEMHVHFKVHGTGKKNLHGDGIALWYTRDRLVPGPVFGSKDNFHGLAIFLDTYPNDETTERVFPYISVMVNNGSLSYDHSKDGRWTELAGCTADFRNRDHDTFLAVRYSRGRLTVMTDLEDKNEWKNCIDITGVRLPTGYYFGASAGTGDLSDNHDIISMKLFQLMVEHTPDEESIDWTKIEPSVNFLKSPKDNVDDPTGNFRSGPLTGWRVFLLLLCALLGIVVCAVVGAVVFQKRQERNKRFY

SEQ ID NO: 22 - protein, 128 aa, Homo sapiens
MGTSSIFLCVLFLCGALGLTMSPARGRLRCYICGFTKPCHPVPTECRDDEACGISIGTSVKKLPLKGPVPSARLCHLLAALLHSVAPLLRAGPVQHSRFPTAAHQPPRLPAPLCGQLRWERTPPPLAS

SEQ ID NO: 23 - protein, 170 aa, Homo sapiens
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSEQSL

SEQ ID NO: 24 - protein, 297 aa, Homo sapiens
MDAEGLALLLPPVTLAALVDSWLREDCPGLNYAALVSGAGPSQAALWAKSPGVLAGQPFFDAIFTQLNCQVSWFLPEGSKLVPVARVAEVRGPAHCLLLGERVALNTLARCSGIASAAAAAVEAARGAGWTGHVAGTRKTTPGFRLVEKYGLLVGGAASHRYDLGGLVMVKDNHVVAAGGVEKAVRAARQAADFALKVEVECSSLQEAVQAAEAGADLVLLDNFKPEELHPTATVLKAQFPSVAVEASGGITLDNLPQFCGPHIDVISMGMLTQAAPALDFSLKLFAKEVAPVPKIH

SEQ ID NO: 25 - protein, 420 aa, Homo sapiens [sequence omitted]

SEQ ID NO: 26 - protein, 373 aa, Homo sapiens
MSGEDEQQEQTIAEDLVVTKYKMGGDIANRVLRSLVEASSSGVSVLSLCEKGDAMIMEETGKIFKKEKEMKKGIAFPTSISVNNCVCHFSPLKSDQDYILKEGDLVKIDLGVHVDGFIANVAHTFVVDVAQGTQVTGRKADVIKAAHLCAEAALRLVKPGNQNTQVTEAWNKVAHSFNCTPIEGMLSHQLKQHVIDGEKTIIQNPTDQQKKDHEKAEFEVHEVYAVDVLVSSGEGKAKDAGQRTTIYKRDPSKQYGLKMKTSRAFFSEVERRFDAMPFTLRAFEDEKKARMGVVECAKHELLQPFNVLYEKEGEFVAQFKFTVLLMPNGPMRITSGPFEPDLYKSEMEVQDAELKALLQSSASRKTQKKKKKK

SEQ ID NO: 27 - protein, 189 aa, Homo sapiens
  MISC_FEATURE (106)..(106): X can be any naturally occurring amino acid
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAIXAGPTALLAHEIGFGSKVTTHPLAKDKMMNGGHYTYSENRVEKDGLILTSRGPGTSFEFALAIVEALNGKEVAAQVKAPLVLKD

SEQ ID NO: 28 - protein, 356 aa, Homo sapiens
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAVGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 29 - protein, 873 aa, Homo sapiens
MATFISMQLKKTSEVDLAKPLVKFIQQTYPSGGEEQAQYCRAAEELSKLRRAAVGRPLDKHEGALETLLRYYDQICSIEPKFPFSENQICLTFTWKDAFDKGSLFGGSVKLALASLGYEKSCVLFNCAALASQIAAEQNLDNDEGLKIAAKHYQFASGAFLHIKETVLSALSREPTVDISPDTVGTLSLIMLAQAQEVFFLKATRDKMKDAIIAKLANQAADYFGDAFKQCQYKDTLPKYFYFQEVFPVLAAKHCIMQANAEYHQSILAKQQKKFGEEIARLQHAAELIKTVASRYDEYVNVKDFSDKINRALAAAKKDNDFIYHDRVPDLKDLDPIGKATLVKSTPVNVPISQKFTDLFEKMVPVSVQQSLAAYNQRKADLINRSIAQMREATTLANGVLASLNLPAAIEDVSGDTVPQSILTKSRSVIEQGGIQTVDQLIKELPELLQRNREILDESLRLLDEEEATDNDLRAKFKERWQRTPSNELYKPLRAEGTNFRTVLDKAVQADGQVKECYQSHRDTIVLLCKPEPELNAAIPSANPAKTMQGSEVVNVLKSLLSNLDEVKKEREGLENDLKSVNFDMTSKFLTALAQDGVINEEALSVTELDRVYGGLTTKVQESLKKQEGLLKNIQVSHQEFSKMKQSNNEANLREEVLKNLATAYDNFVELVANLKEGTKFYNELTEILVRFQNKCSDIVFARKTERDELLKDLQQSIAREPSAPSIPTPAYQSSPAGGHAPTPPTPAPRTMPPTKPQPPARPPPPVLPANRAPSATAPSPVGAGTAAPAPSQTPGSAPPPQAQGPPYPTYPGYPGYCQMPMPMGYNPYAYGQYNMPYPPVYHQSPGQAPYPGPQQPSYPFPQPPQQSYYPQQ

SEQ ID NO: 30 - protein, 423 aa, Homo sapiens [sequence omitted]

SEQ ID NO: 31 - protein, 164 aa, Homo sapiens
GKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 32 - protein, 310 aa, Homo sapiens
RSGLWVFCYRDRLVMQKGLKDNFADVQVSVVDCPDLTKEPFTFPVKGICGKTRIAEVGGVPYLLPLVNQKKVYDLNKIAKEIKLPGAFILGAGAGPFQTLGFNSEFMPVIQTESEHKPPVNGSYFAHVNPADGGCLLEKYSEKCHDFQCALLANLFASEGQPGKVIEVKAKRRTGPLNFVTCMRETLEKHYGNKPIGMGGTFIIQKGKVKSHIMPAEFSSCPLNSDEEVNKWLHFYEMKAPLVCLPVFVSRDPGFDLRLEHTHFFSRHGEGGHYHYDTTPDIVEYLGYFLPAEFLYRIDQPKETHSIGRD

SEQ ID NO: 33 - protein, 251 aa, Homo sapiens
QNDLRPLLIERYPGSPGSYAARQHIMQRIQRLQADWVLEIDTFLSQTPYGYRSFSNIISTLNPTAKRHLVLACHYDSKYFSHWNNRVFVGATDSAVPCAMMLELARALDKKLLSLKTVSDSKPDLSLQLIFFDGEEAFLHWSPQDSLYGSRHLAAKMASTPHPPGARGTSQLHGMDLLVLLDLIGAPNPTFPNFFPNSARWFERLQAIEHELHELGLLKDHSLEGRYFQNYSYGGVIQDDHIPFLRRGVPV

SEQ ID NO: 34 - protein, 472 aa, Homo sapiens
MATKCGNCGPGYSTPLEAMKGPREEIVYLPCIYRNTGTEAPDYLATVDVDPKSPQYCQVIHRLPMPNLKDELHHSGWNTYSSCFGDSTKSRNKLVLPSLISSRIYVVDVGSEPGPQKLHKVIEPKDIHAKCELACLHTSHCLASGEVMISSLGDVKGNGKGGFVLLDGETFEVKGTWERPGGAAPLGYDFWYQPRHNVMISTEWAAPNVLRDGFNPADVEAGLYGSHLYVWDWQRHEIVQTLSLKDGLIPLEIRFLHNPSATQGFVGCASAPNIQRFYKTREGTWSVEKVIQVPPKKVKGWLLPGVPGLITDILLSLDDRFLYFSNWLHGDLRQYDISDPQRPRLTGQLFLGGSIVKGGPVQVLEDEELKSQPEPLVVKGKRVAGGPQMIQLSLDGKRLYITTSLYSAWEKQFYPDLIREGSVMLQVDVDTVKGGLKLNPNCLVDFGKEPLGPALAHELRYPGGDCSSDIWI

SEQ ID NO: 35 - protein, 543 aa, Homo sapiens
MEQVNELKEKGNKALSVGNIDDALQCYSEAIKLDPHNHVLYSNRSAAYAKKGDYQKAYEDGCKTVDLKPDWGKGYSRKAAALEFLNRFEEAKRTYEEGLKHEANNPQLKEGLQNMEARLAERKFMNPFNMPNLYQKLESDPRTRTLLSDPTYRELIEQLRNKPSDLGTKLQDPRIMTTLSVLLGVDLGSMDEEEEIATPPPPPPPKKETKPEPMEEDLPENKKQALKEKELGNDAYKKKDFDTALKHYDKAKELDPTNMTYITNQAAVYFEKGDYNKCRELCEKAIEVGRENREDYRQIAKAYARIGNSYFKEEKYKDAIHFYNKSLAEHRTPDVLKKCQQAEKILKEQERLAYINPDLALEEKNKGNECFQKGDYPQAMKHYTEAIKRNPKDAKLYSNRAACYTKLLEFQLALKDCEECIQLEPTFIKGYTRKAAALEAMKDYTKAMDVYQKALDLDSSCKEAADGYQRCMMAQYNRHDSPEDVKRRAMADPEVQQIMSDPAMRLILEQMQKDPQALSEHLKNPVIAQKIQKLMDVGLIAIR

SEQ ID NO: 36 - protein, 318 aa, Homo sapiens
MSSSPVKRQRMESALDQLKQFTTVVADTGDFHAIDEYKPQDATTNPSLILAAAQMPAYQELVEEAIAYGRKLGGSQEDQIKNAIDKLFVLFGAEILKKIPGRVSTEVDARLSFDKDAMVARARRLIELYKEAGISKDRILIKLSSTWEGIQAGKELEEQHGIHCNMTLLFSFAQAVACAEAGVTLISPFVGRILDWHVANTDKKSYEPLEDPGVKSVTKIYNYYKKFSYKTIVMGASFRNTGEIKALAGCDFLTISPKLLGELLQDNAKLVPVLSAKPKPVTWKKSTWMRSLSVGCTTRTRWLWRSSLTGSASLPLMQ

SEQ ID NO: 37 - protein, 534 aa, Homo sapiens
MPYEIKKVFASLPQVERGVSKIIGGDPKGNNFLYTNGKCVILRNIDNPALADIYTEHAHQVVVAKYAPSGFYIASGDVSGKLRIWDTTQKEHLLKYEYQPFAGKIKDIAWTEDSKRIAVVGEGREKFGAVFLWDSGSSVGEITGHNKVINSVDIKQSRPYRLATGSDDNCAAFFEGPPFKFKFTIGDHSRFVNCVRFSPDGNRFATASADGQIYIYDGKTGEKVCALGGSKAHDGGIYAISWSPDSTHLLSASGDKTSKIWDVSVNSVVSTFPMGSTVLDQQLGCLWQKDHLLSVSLSGYINYLDRNNPSKPLHVIKGHSKSIQCLTVHKNGGKSYIYSGSHDGHINYWDSETGENDSFAGKGHTNQVSRMTVDESGQLISCSMDDTVRYTSLMLRDYSGQGVVKLDVQPKCVAVGPGGYAVVVCIGQIVLLKDQRKCFSIDNPGYEPEVVAVHPGGDTVAIGGVDGNVRLYSILGTTLKDEGKLLEAKGPVTDVAYSHDGAFLAVCDASKVVTVFSVADGYSENNVFYGHHEK

SEQ ID NO: 38 - protein, 511 aa, Homo sapiens
MKFFLLLFTIGFCWAQYSPNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMSGNAVSAGTSSTCGSYFNPGSRDFPAVPYSGWDFNDGKCKTGSGDIENYNDATQVRDCRLVGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 39 - protein, 511 aa, Homo sapiens
MKFFLLLFTIGFCWAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 40 - protein, 496 aa, Homo sapiens
  MISC_FEATURE (1)..(1): X can be any naturally occurring amino acid
XYSSNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLSGLLDLALGKDYVRSKIAEYMNHLIDIGVAGFRIDASKHMWPGDIKAILDKLHNLNSNWFPEGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRYFENGKDVNDWVGPPNDNGVTKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 41 - protein, 249 aa, Homo sapiens
RHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 42 - protein, 285 aa, Homo sapiens
STQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 43 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 44 - protein, 182 aa, Homo sapiens
ERDCRVSSFRVKENFDKARFSGTWYAMAKKDPEGLFLQDNIVAEFSVDETGQMSATAKGRVRLLNNWDVCADMVGTFTDTEDPAKFKMKYWGVASFLQKGNDDHWIVDTDYDTYAVQYSCRLLNLDGTCADSYSFVFSRDPNGLPPEAQKIVRQRQEELCLARQYRLIVHNGYCDGRSERNL

(19) United States
(12) Patent Application Publication
(10) Pub. No.: US 2009/0209431 A1
(43) Pub. Date: Aug. 20, 2009

(54) Non-Invasive in Vitro Method to Detect Transitional Cell Carcinoma of the Bladder

(75) Inventors: Ana Soloaga Villoch, Bilbao (ES); Jose Luis Castrillo Diez, Getxo (ES); Inma Ramos Rodriguez, Bilbao (ES); Kepa Escuredo Garcia-Galdeano, Bilbao (ES); Antonio Martinez Martinez, Laukariz (ES); Laureano Simon Buela, San Sebastian (ES)

Correspondence Address: WOLF GREENFIELD & SACKS, P.C., 600 ATLANTIC AVENUE, BOSTON, MA, 02210-2206, US

(73) Assignee: LABORATORIOS SALVAT, S.A., Esplugues del Llobregat (ES)

(21) Appl. No.: 12/083,446

(22) PCT Filed: Oct. 10, 2006
(86) PCT No.: PCT/EP2006/009768

§ 371 Date: Mar. 12, 2009

(30) Foreign Application Priority Data
Oct. 11, 2005 (EP) 05384037.7

(51) Int. Cl. C40B 30/00; G01N 33/574; G01N 33/53; C12Q 1/02

(52) U.S. Cl. 506/7; 436/64; 436/501; 435/7.92; 435/29

(57) ABSTRACT

The present invention refers to a non-invasive in vitro method to detect a transitional cell carcinoma of the bladder in an individual via urine analysis, to determine the stage or severity of this cancer in an individual or to monitor the effect of treatment administered to an individual suffering from this cancer.

### FIELD OF THE INVENTION

The present invention refers to a non-invasive in vitro method to detect the presence of transitional cell carcinoma of the bladder in the urine of an individual via urine analysis, as well as to the use of peptide sequences derived from selected proteins and to a kit to perform the method.

### BACKGROUND OF THE INVENTION

Bladder cancer is the most common cancer of the urinary tract; it is also the fourth most common cancer in men and the eighth most common in women. It includes a broad spectrum of tumors of various histological types including transitional cell carcinoma of the bladder (BTCC, 90%), squamous cell carcinoma (7%), adenocarcinoma (2%), and undifferentiated carcinoma (1%).

Tumor grade and stage are the best prognostic indicators of BTCC. Bladder tumors are graded cytomorphologically from G1 (low grade) to G3 (high grade) in decreasing state of differentiation and increasing aggressiveness of the disease according to the World Health Organization (WHO). With respect to stage or invasiveness, BTCCs are classified as superficial papillary (Ta and T1), muscle invasive (T2 to T4), or the uncommon carcinoma in situ or tumor in situ (TIS).

Low-grade tumors are usually confined to the mucosa or infiltrate superficial layers (stage Ta and T1). Most high-grade tumors are detected at least at T1 stage (invading lamina propria). Approximately 75% of the diagnosed bladder cancer cases are superficial. The remaining 25% are muscle invasive at the moment of diagnosis. Patients with superficial BTCC have a good prognosis but have a 70% risk of recurrence. In spite of this high risk, Ta tumors tend to be low grade and only 10-15% will progress to muscle invasion in 2 years. However, the percentage of T1 tumors that progresses to T2 stage is higher (30-50%).

Currently, the best diagnostic system for bladder cancer in individuals is established either by cystoscopy and transurethral biopsy or by resection, both representing invasive procedures.

Flexible cystoscopes make the technique less aggressive, but it remains invasive and highly unpleasant, still requiring some form of anaesthesia.

The prevailing non-invasive technique for diagnosis of BTCC is to identify neoplastic cells by morphological examination of the cells in urine. Thus, cytology is currently used to monitor patients diagnosed with and treated for bladder cancer. Although urine cytology can detect tumors in situ that are not detectable by cystoscopy, as well as tumors located in the upper end of the bladder or the upper urinary tract, i.e. ureter, pelvis and kidney, several studies have shown that cytology has a very low sensitivity in bladder cancer diagnosis, missing up to 50% of tumors. Findings from cytologies are subtle and can be confused with reactive or degenerative processes. Cytology studies require expert, individual evaluation, which delays the availability of the results and further introduces subjectivity and variance to the final results. Actually, there is no highly sensible and specific non-invasive method available to diagnose bladder cancer (Boman H. et al., J Urol 2002, 167:80-83).

Many efforts have been made to improve non-invasive cancer detection. Recent advances in expression profiling of cancer cells by proteomic technologies, high resolution two dimensional electrophoresis and mass spectrometry have made it possible to identify candidate proteins as tumor markers for bladder cancer, such as nuclear matrix protein NMP22 (Soloway M. S. et al., J Urol 1996, 156:363-367), hyaluronic acid and hyaluronidase (Pham H. T. et al., Cancer Res 1997, 57:778-783), basement membrane complexes (BTA, Pode D. et al., J Urol 1999, 161:443-446), carcinoembryonic antigen (CEA, Halim A. B. et al., Int J Biol Markers, 1992; 7:234-239), uroplakin II (Wu X. R. et al., Cancer Res 1998; 58:1291-1297), scatter factor/hepatocyte growth factor (SF/HGF, Gohji K. et al., J Clin Oncol 2000; 18:2963-2971), proteins of the keratin/cytokeratin family like cytokeratin 20 (Buchumensky V. et al., J Urol 1998, 160:1971-1974), cytokeratin 18 (Sánchez-Carbayo M. et al., Clin Cancer Res 2000, 6:3585-3594), mammary tumor 8-Ka protein (MAT-8, Morrison B. W. et al., J Biol Chem 1995, 270:2176-2182) and telomerase (Lee D. H. et al., Clinical Cancer Research 1998, 4: 535-538).

Memon A. A. et al. (Cancer Detect Prev 2005, 29:249-255) identify seven differentially expressed proteins in bladder cancer from cell lines and biopsies. However no control is used for comparison. Langbein, S. et al (Technol Cancer Res Treat 2006, 67-71) disclose the protein profiling of biopsies from bladder cancer patients using the 2D-PAGE and SELDI-TOF-MS technique.

Rasmussen H. H. et al. (J Urol 1996, 155:2113-2119) disclose a database of the most abundant proteins present in the urine of patients with bladder cancer. Celis J. E. et al (Electrophoresis 1999, 300-309) describe a database of proteins present in biopsies from bladder cancer patients. Nevertheless, no markers are identified in any of these references, since the authors do not show a quantified differential profile of the proteins in healthy samples vs tumoral samples.

Further, no marker to predict the prognosis and extent of bladder cancer has been proven useful in clinical trials (Miyake H. et al., J Urol 2002, 167:1282-1287).

### DESCRIPTION OF THE INVENTION

The current invention provides a highly sensitive, efficient and rapid non-invasive in vitro method concerning proteins associated with BTCC. It further concerns a method for the diagnosis, prognosis and monitoring of BTCC via analysis of urine samples. The current invention surprisingly provides bladder cancer biomarkers for detection of BTCC via urine analysis. A preferred embodiment of the invention concerns 40 bladder cancer biomarkers for detection of BTCC via urine analysis that are of significant value for the detection, diagnosis, prognosis and/or monitoring of BTCC.

Aminoacylase-1 (ACYL) is a cytosolic, homodimeric, zinc-binding enzyme that catalyzes the hydrolysis of acylated L-amino acids to L-amino acids and acyl group, and has been postulated to function in the catabolism and salvage of acylated amino acids. ACY1 has been assigned to chromosome 3p21.1, a region reduced to homozygosity in small-cell lung cancer (SCLC), and its expression has been reported to be reduced or undetectable in SCLC cell lines and tumors. ACY1 is the first member of a new family of zinc-binding enzymes.

Aldehyde reductase (AKR1A1) belongs to the aldo/keto reductase family, it is involved in the reduction of biogenic and xenobiotic aldehydes and is present in virtually every tissue.

Aldolase B (ALDOB), also called fructose 1-phosphate aldolase, is produced in the liver, kidneys and brain. It is needed for the breakdown of fructose.

Alpha-amylase (AMY) carcinoid, pancreatic alpha-amylase and alpha amylase 1A belong to the glycosyl hydrolase 13 family and hydrolyze 1,4-alpha-glucoside bonds in oligosaccharides and polysaccharides, and thus catalyze the first step in digestion of dietary starch and glycogen. The human genome has a cluster of several amylase genes that are expressed at high levels in either salivary gland or pancreas.

Annexin IV (ANXA4) belongs to the annexin family of calcium-dependent phospholipid binding proteins. Although their functions are still not clearly defined, several members of the annexin family have been implicated in membrane-related events along exocytotic and endocytotic pathways. ANXA4 is almost exclusively expressed in epithelial cells.

Apolipoprotein A-I (APOA1) belongs to the apolipoprotein A1/A4/E family and participates in the reverse transport of cholesterol from tissues to the liver for excretion by promoting cholesterol efflux from tissues and by acting as a cofactor for the lecithin cholesterol acyltransferase (LCAT).

Biliverdin reductase A (BIEA) belongs to the gfo/idh/moca family and biliverdin reductase subfamily and reduces the gamma-methene bridge of the open tetrapyrrole, biliverdin IX alpha, to bilirubin with the concomitant oxidation of NADH or NADPH.

Flavin reductase (BLVRB) catalyzes electron transfer from reduced pyridine nucleotides to flavins as well as to methylene blue, pyrroloquinoline quinone, riboflavin, or methemoglobin. BLVRB has a possible role in protecting cells from oxidative damage or in regulating iron metabolism. In the liver, it converts biliverdin to bilirubin. It is predominantly expressed in liver and erythrocytes, and at lower levels in heart, lung, adrenal gland and cerebrum.

Cathepsin D (CATD) is an acid protease which belongs to the peptidase A1 family and activates intracellular protein breakdown. It is involved in the pathogenesis of several diseases such as bladder cancer (Ozer E., et al., Urology 1999, 54:50-5 and Ioachim E., et al., Anticancer Res 2002, 22:3383-8) breast cancer and possibly Alzheimers disease. Cathepsin D is synthesized as an inactive precursor of 52 kDa, formed of two polypeptides of 14 kDa (CATD H) and one of 34 kDa (CATD K). The inactive peptide is activated by proteolysis of the N-terminus resulting in the enzymatically active protein of 48 kDa

Complement C3 precursor (CO3) plays a central role in the activation of the complement system. Its processing by C3 convertase is the central reaction in both complement pathways. CO3 has been related to urogenital tumors (Dunzendorfer U., et al., Eur Urol 1980, 6:232-6).

Cytosolic nonspecific dipeptidase (CPGL1) belongs to the peptidase M20A family and is also known as tissue camosinase and peptidase A. It is a nonspecific dipeptidase rather than a selective camosinase.

Alpha enolase (ENOA) is a multifunctional enzyme that, as well as its role in glycolysis, plays a part in various processes such as growth control, hypoxia tolerance and allergic responses. It may also function in the intravascular and pericellular fibrinolytic system due to its ability to serve as a receptor and activator of plasminogen on the cell surface of several cell types such as leukocytes and neurones. Mammalian enolase is composed of 3 isozyme subunits, alpha, beta and gamma, which can form homodimers or heterodimers which are cell type and development-specific. ENOA interacts with PLG in the neuronal plasma membrane and promotes its activation. It is used as a diagnostic marker for many tumors and, in the heterodimeric form, alpha/gamma, as a marker for hypoxic brain injury after cardiac arrest. It has been related to bladder cancer (Iczkowski K. A., et al., Histopathology 1999, 35:150-6).

Ferritin light chain (FRIL) belongs to the ferritin family and is the major intracellular iron storage protein in prokaryotes and eukaryotes. It is composed of 24 subunits of the heavy and light ferritin chains. Variation in ferritin subunit composition may affect the rates of iron uptake and release in different tissues. A major function of ferritin is the storage of iron in a soluble and non-toxic state.

Rab GDP dissociation inhibitor beta (GDIB) belongs to the rab gdi family and regulates the GDP/GTP exchange reaction of members of the rab family, small GTP-binding proteins of the ras superfamily, that are involved in vesicular trafficking of molecules between cellular organelles. GDIB is ubiquitously expressed. Plasma glutathione peroxidase (GPX3) belongs to the glutathione peroxidase family and catalyzes the reduction of hydrogen peroxide, organic hydroperoxide, and lipid peroxides by reduced glutathione and functions in the protection of cells against oxidative damage. Human plasma glutathione peroxidase has been shown to be a selenium-containing enzyme. GPX3 expression appears to be tissue-specific.

Gluthatione synthetase (GSHB) is important for a variety of biologic functions, including protection of cells from oxidative damage by free radicals, detoxification of xenobiotics and membrane transport.

Gelsolin (GSN40 and GSN80) is a calcium-regulated, actin-modulating protein that binds to the plus ends of actin monomers or filaments, preventing monomer exchange (end-blocking or capping). It can promote the assembly of monomers into filaments (nucleation), as well as sever existing filaments. In addition, this protein binds to actin and to fibronectin. It has been related to bladder cancer, although it has not been described as a biomarker, that is, its expression level has not been compared and quantified in healthy individuals vs cancer patients (Rasmussen, H. H. et al., J Urol 1996, 155:2113-2119; Haga K, Hokkaido Igaku Zasshi 2003, 78:29-37; Celis A et al., Electrophoresis 1999, 20:355-61).

Glutathione S-transferase (GSTP1) belongs to a family of enzymes that play an important role in detoxification by catalysing the conjugation of many hydrophobic and electrophilic compounds with reduced glutathione.

Cytoplasmic isocitrate dehydrogenase (IDHC) belongs to the isocitrate and isopropylmalate dehydrogenase family and catalyzes the oxidative decarboxylation of isocitrate to form alpha-ketoglutarate.

Vesicular integral-membrane protein VIP36 precursor (LMAN2) plays a role as an intracellular lectin in the early secretory pathway. Interacts with N-acetyl-D-galactosamine and high-mannose type glycans and may also bind to O-linked glycans. It is involved in the transport and sorting of glycoproteins carrying high mannose-type glycans.

Lymphocyte antigen 6 complex, locus 6 G6E (LY6G6E) belongs to the Lymphocyte antigen-6 superfamily. Members of this family are cystein-rich, generally GPI-anchored cell surface proteins, which have definite or putative immune related roles. Its specific function is unknown. Mannan-binding lectin serine protease 2, precursor (MASP2) belongs to the peptidase s1 family and is a trypsin protease that presumably plays an important role in the initiation of the mannose binding lectin (MBL) complement activation pathway. After activation it cleaves C4 generating C4A and C4B.

Nicotinate-nucleotide pyrophosphorylase (NADC) belongs to the nadC/mod D family and is an intermediate in the de novo synthesis pathway of NAD from tryptophan and acts as a potent endogenous excitotoxin through hyperstimulation of N-methyl D-aspartate (NMDA) receptor in the neuron. Elevation of NADC levels in the brain has been linked to the pathogenesis of neurodegenerative disorders such as epilepsy, Alzheimer's disease and Huntington's disease. It has not been related to cancer yet.

Napsin A (NAPSA) belongs to the peptidase al family and it is expressed at the highest levels in the kidney, at a moderate level in the lung, and at low levels in the spleen and adipose tissue. It may be involved in processing of pneumocyte surfactant precursors.

Proliferation-associated protein 2G4 (PA2G4) is a single-stranded DNA-binding protein showing cell cycle specific variation in nuclear localization, which belongs to the peptidase m24c family. As the protein is expressed in response to mitogen stimulation, it may belong to a large family of cell cycle regulatory proteins or replication proteins that maintain the cell cycle activities of proliferating cells.

Oncogene DJ1 (PARK7) is highly expressed in pancreas, kidney, skeletal muscle, liver, testis and heart and acts as positive regulator of androgen receptor-dependent transcription. It may function as redox-sensitive chaperone and as sensor for oxidative stress. It prevents aggregation of SNCA and protects neurons against oxidative stress and cell death.

Poly(rc)-binding protein 1 (PCPB1) is a single stranded nucleic acid binding protein that binds preferentially to oligo dC. It may shuttle between the nucleus and the cytoplasm. It is abundantly expressed in skeletal muscle, thymus and peripheral blood leucocytes while a lower expression is observed in prostate, spleen, testis, ovary, small intestine, heart, liver, adrenal and thyroid glands.

Programmed cell death 6-interacting protein (PDCB1) is expressed in brain, heart, placenta, lung, kidney, pancreas, liver, skeletal muscle and cochlea and may play a role in the regulation of both apoptosis and cell proliferation. It interacts with PDCD6/ALG-2 and SH3 KBP1.

Lysosomal acid phosphatase precursor (PPAL) belongs to the histidine acid phophatase family and hydrolyzes orthophosphoric monoesters to alcohol and phosphate. Peroxiredoxin 2 (PRDX2) belongs to the ahpc/tsa family and is involved in redox regulation of the cell. It reduces peroxides with reducing equivalents provided through the thioredoxin system. It is not able to receive electrons from glutaredoxin. It may play an important role in eliminating peroxides generated during metabolism. It might participate in the signalling cascade of growth factors and tumor necrosis factor-alpha by regulating the intracellular concentrations of H₂O₂. It enhances natural killer (NK) cells activity.

Glutaminyl-peptide cyclotransferase (QPCT) is responsible for the biosynthesis of pyroglutamyl peptides. It has a bias against acidic and tryptophan residues adjacent to a N-terminal glutamic acid.

Plasma retinol-binding protein (RETBP) belongs to the lipocalin family and is the specific carrier for retinol (vitamin A alcohol) in the blood. It delivers retinol from the liver stores to the peripheral tissues. In plasma, the RBP-retinol complex interacts with transthyretin, which prevents its loss by filtration through the kidney glomeruli. A deficiency of vitamin A blocks secretion of the binding protein posttranslationally and results in defective delivery and supply to the epidermal cells.

Selenium-binding protein (SBP1) belongs to the selenium-binding protein family. Selenium is an essential nutrient that exhibits potent anticarcinogenic properties; a deficiency in selenium may cause certain neurologic diseases. It has been proposed that the effects of selenium in preventing cancer and neurologic diseases may be mediated by selenium-binding proteins.

Stress-induced-phosphoprotein 1 (STIP1) is an adaptor protein that mediates the association of the molecular chaperones HSC70 and HSP90 (HSPCA and HSPCB).

Transaldolase (TALDO) belongs to the transaldolase family and is a key enzyme of the nonoxidative pentose phosphate pathway providing ribose-5-phosphate for nucleic acid synthesis and NADPH for lipid biosynthesis. This pathway can also maintain glutathione at a reduced state and thus protect sulfhydryl groups and cellular integrity from oxygen radicals.

Transthyretin (TTHY, formerly prealbumin) is one of 3 thyroid hormone-binding proteins found in the blood of vertebrates. It is produced in the liver and circulates in the bloodstream, where it binds retinol and thyroxine.

WD repeat protein 1 (WDR1) belongs to the wd-repeat aip1 family and induces dissassembly of actin filaments in conjunction with ADF/cofilin family proteins.

Cancer can be detected by analysing the expression pattern of at least one bladder cancer biomarker in a test urine sample. Thus, detection of at least one differentially expressed protein in a urine test sample in comparison to normal urine is indicative of BTCC.

Urine samples are very diverse in composition. Therefore, normalization is essential to account for differences in total protein concentration and to remove bias from sample to sample.

The expression levels of a control protein, whose content in urine is always constant, may be used to normalize signal levels. In the literature there are many examples of these non-variable proteins. In the present invention, transferrin was shown to be a non-variable protein.

Representative bladder cancer proteins or biomarkers identified in the present invention include, but are not limited to TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO, WDR1, AMY, APOA1, GSN40, GSN80 and RETBP (also referred as to the 40 bladder cancer biomarkers).

One aspect of the present invention relates to a non-invasive in vitro method that comprises: a) detecting and quantifying one or more biomarkers present in a urine test sample from an individual; and b) comparing the expression measurement obtained in a) in the test urine sample to the corresponding standard value in normal urine, wherein variations in the measurement obtained in a) compared to the corresponding standard value in normal urine are indicative of BTCC. This method can be adapted for screening to detect the presence of BTCC, to determine the stage or severity of this cancer.

In addition, this method may also be employed to assess the lack of disease after surgical resection, to establish the diagnosis and/or prognosis of this cancer and/or to monitor the effect of the treatment administered to an individual suffering from said cancer.

Another aspect of the invention relates to the use of one or more peptide sequences derived from biomarkers present in urine to detect the presence of BTCC via urine analysis, to determine the stage or severity of this cancer, to assess the lack of disease after surgical resection, to establish the diagnosis and/or prognosis of this cancer and/or to monitor the effect of the treatment administered to an individual suffering from said cancer.

Another aspect of the invention relates to the use of one or more nucleotides or peptide sequences derived from the 40 bladder cancer biomarkers or a transcriptional or a post-translational variant thereof, alone or in any combination, in methods to screen for, identify, develop and evaluate the efficacy of therapeutic agents to treat BTCC.

The invention additionally provides antibodies directed against such bladder cancer biomarkers. These antibodies may be provided in a variety of forms, as appropriate for a particular use, including, for example, in a soluble form, immobilized on a substrate, or in combination with a pharmaceutically acceptable carrier.

Another aspect of the invention relates to a kit to perform the method as previously defined comprising 1) at least one antibody that specifically recognises one cancer biomarker in urine and 2) a carrier in a suitable packaging.

Another aspect of the invention relates to a BTCC reference expression profile, comprising a pattern of expression of one or more bladder cancer biomarkers. Different BTCC reference expression profiles may be established and correlated to different cancer stages.

For the purposes of the present invention the following definitions have been used:

The term “cancer” refers to the disease that is typically characterised by abnormal or unregulated cell growth, capable of invading adjacent tissues and spreading to distant organs. The term “carcinoma” refers to the tissue resulting from abnormal or unregulated cell growth. The term “transitional cell carcinoma of the bladder” or its abbreviation “BTCC” refer to any malign proliferative disorder in bladder epithelial cells. The term “tumor” refers to any abnormal mass of tissue generated by a neoplastic process, whether this is benign (non cancerous) or malignant (cancerous).

The term “bladder cancer proteins or biomarkers” refers to differentially expressed proteins in BTCC, that is, proteins which are expressed differently in the urine from a healthy individual as compared to the urine from a patient having BTCC. In the present invention the group of proteins comprised by differentially expressed proteins includes both the 40 biomarkers selected from the group comprising TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO, WDR1, AMY, APOA1, GSN40, GSN80 and RETBP, as well as any protein of a urine sample, whose ratio changes at least two-fold when comparing two different urine samples: one from a healthy individual and one from a patient of transitional cell carcinoma, this quantification being carried out by an image analyzer software, for example Progenesis PG220 software. Bladder cancer biomarkers as described herein may be of any length and further comprise additional sequences derived from the native protein and/or heterologous sequences, any transcriptional or post-translational variants, as well as any sequence with at least 95% identity with the described sequences, wherein identity is defined as the percentage of residues which are identical between two sequences. Those skilled in the art will appreciate that these other portions or variants may be also useful in the treatment and detection of cancer.

The term “detecting one or more biomarkers” means determining the existence of one or more biomarkers, whereas the term “quantifying one or more biomarkers” means expressing the presence of one or more biomarkers as a value (for instance as an intensity value). The term “indicative of BTCC” means that variations found in the measurement of one or more biomarkers in a test urine sample as compared to the corresponding standard value in normal urine serve as a proof of the presence of BTCC. The term “variation” refers to a change in the level of expression of a protein.

The term “differentially expressed protein” refers to a protein, whose expression pattern varies (increases or decreases) in the urine from a patient having BTCC as compared to the urine from a healthy individual. The term “inversely expressed” refers to two differentially expressed proteins whose expression patterns are opposite (that is, in a sample one is overexpressed while the other is repressed or vice versa).

The term “protein extract” corresponds to the supernatant obtained after centrifugation of the urine sample.

The term “individual” refers to all species of animals classified as mammals and includes, but is not restricted to, domestic and farm animals, primates and humans, and preferably refers to a male or female human of any age or race. The term “healthy individual” refers to an individual who does not suffer from BTCC and may include patients having other urologic diseases. The term “previously diagnosed” relates to an individual who has already received a first positive diagnostic for BTCC. The term “not previously diagnosed” relates to an individual who has never received a first positive diagnostic for BTCC (de novo diagnostic).

The term “standard value in normal urine” relates to the quantification of the average expression level of one or more biomarkers detected in single urine samples of individuals known to not be suffering from BTCC.

The term “diagnosis” of BTCC relates to the process of identifying or determining the nature and cause of BTCC through evaluation of one or more BTCC biomarkers. The term “prognosis” refers to the likely outcome or course of a disease, that is the chance of recovery or recurrence. The term “monitoring” means to assess the presence or absence of BTCC in an individual at different times. The term “treatment” refers to any process, action, application or the like, wherein an individual is subject to medical aid with the object of improving his condition, directly or indirectly.

The term “specificity” refers to the ability of a test to exclude the presence of a disease when it is truly not present. Specificity is expressed as the number of healthy individuals for whom there is a correct negative test (this group being called true negatives), divided by the sum of true negatives and the number of healthy individuals for whom there is an incorrect positive test (this group being called false positives). The term “sensitivity” refers to the ability of a test to detect a disease when it is truly present. Sensitivity is expressed as the number of diseased patients for whom there is a correct positive test (this group being called true positives), divided by the sum of true positives and the number of diseased patients for whom there is an incorrect negative test (this group being called false negatives). The term “robustness” defines the ability of the numerical method to provide the same result despite variabilities in the initial samples.

The term “gene” refers to a region of a molecular chain of deoxyribonucleotides that encodes a protein and may represent a portion of a coding sequence or a complete coding sequence. The term “protein” refers to at least one molecular chain of amino acids linked intermolecularly through either covalent or non-covalent bonds. The term includes all forms of post-translational protein modifications, for example glycosylation, phosphorylation or acetylation. The terms “peptide” and “polypeptide” refer to molecular chains of amino acids that represent a protein fragment. The terms “protein” and “peptide” are indistinguishable.

The term “antibody” refers to a Y-shaped protein (known as immunoglobulin) on the surface of B cells that is secreted into the blood or lymph in response to an antigenic stimulus, such as an exogenous protein, bacterium, virus, parasite, or transplanted organ, and that exhibits a specific binding activity for a target molecule called an “antigen”. The antigen binding region of immunoglobulins can be divided into either F(ab′)₂ or Fab fragments. The term “antibody” includes monoclonal and polyclonal antibodies, either intact or fragments derived from them; and includes human antibodies, humanized antibodies and antibodies of non-human origin. A “non-human antibody” is an antibody generated by an animal species other than Homo sapiens. A “humanized antibody” is a genetically engineered antibody in which the minimal mouse part from a murine antibody is fused to a human antibody. Generally, humanized antibodies are 5-10% mouse and 90-95% human. A “human antibody” is an antibody derived from a transgenic mice carrying human antibody genes or from human cells. The “monoclonal antibodies” are homogeneous, highly specific antibody populations directed against a single antigenic site or “determinant” of the target molecule. “Polyclonal antibodies” include heterogeneous antibody populations that are directed against different antigenic determinants of the target molecule. The term “specific antibody” refers to an antibody generated against a specific protein (in this case against a particular bladder cancer marker). The term “antibody-protein complex” refers to a complex formed by an antigen and its specific antibody. The term “combibody” (combinatorial antibody) refers to an antibody displayed on filamentous phages, which allows direct screening of cDNA libraries for expression of cell-surface-reactive antibodies, without the need for antibody production and purification using bacteria or eukaryotic cell systems. The term “Fab recombinant antibody” refers to a recombinant antibody that only contains the Fab fragment that is univalent and useful when the antibody has a very high affinity for its antigen. They can be recombinantly obtained if the protein sequence is known. The term “ScFv antibody fragment” refers to a single chain variable fragment (scFv) that can be expressed in bacterial cultures.

The term “epitope” refers to an antigenic determinant of a protein, which is the sequence of amino acids of the protein that a specific antibody recognises. Such epitopes may be comprised of a contiguous stretch of amino acids (linear epitope) or of non-contiguous amino acids that are brought into proximity with one another by virtue of the three dimensional folding of the polypeptide chain (discontinuous epitopes). The term “solid phase” refers to a non-aqueous matrix to which the antibody can be bound. Examples of materials for the solid phase include but are not limited to glass, polysaccharides (for example agarose), polyacrylamide, polystyrene, polyvinylic alcohol and silicons. Examples of solid phase forms are the well of a plate or a purification column.

The term “dipstick” refers to a device dipped into a liquid to perform some kind of test that may determine and/or quantify some properties of the liquid (chemical, physical, etc). This kind of dipstick is usually made of paper or cardboard and is impregnated with reagents whose colour changes indicate some feature of the liquid. The term “carrier” refers to a mechanism or device by which something is conveyed or conducted. The term “packaging” refers to the containment and packing prior to sale with the primary purpose of facilitating the purchase and use of a product.

The term “biochip” refers to a collection of miniaturized test sites (microarrays) arranged on a solid substrate that permits many tests to be performed at the same time in order to achieve higher throughput and speed.

In one embodiment of the invention, the first step of the method to assess bladder cancer comprises measuring one or more biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO, WDR1, AMY, APOA1, GSN40, GSN80 and RETBP (also referred as to the 40 bladder cancer biomarkers) or a transcriptional or a post-translational variant thereof in a urine test sample from an individual.

In another embodiment, the first step of the method to assess bladder cancer comprises measuring one or more biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO and WDR1 or a transcriptional or a post-translational variant thereof in a urine test sample from an individual.

In another embodiment, the first step of the method to assess bladder cancer comprises measuring at least two biomarkers or a transcriptional or a post-translational variant thereof in the test urine sample. In another embodiment, the first step of the method to assess bladder cancer comprises measuring at least three biomarkers or a transcriptional or a post-translational variant thereof in the test urine sample. In another embodiment, the first step of the method to assess bladder cancer comprises measuring at least four biomarkers or a transcriptional or a post-translational variant thereof in the test urine sample. In another embodiment, the first step of the method to assess bladder cancer comprises measuring at least five biomarkers or a transcriptional or a post-translational variant thereof in the test urine sample.

In another embodiment, the first step of the method to assess bladder cancer comprises measuring one or more biomarkers selected from TTHY, ACY1, AKR1A1, ANXA4, BIEA, BLVRB, CATD H, CATD K, CO3, CPGL1, ENOA, FRIL, GDIB, GPX3, GSHB, GSTP1, IDHC, LY6G6E, MASP2, NADC, NAPSA, PA2G4, PARK7, PCBP1, PRDX2, PTD012, QPCT, SBP1, STIP1, AMY, APOA1, GSN40, GSN80 and RETBP or a transcriptional or a post-translational variant thereof in the test urine sample.

In another embodiment, the first step of the method to assess bladder cancer comprises measuring one or more biomarkers selected from TTHY, ACY1, AKR1A1, ANXA4, BIEA, BLVRB, CATD H, CATD K, CO3, CPGL1, ENOA, FRIL, GDIB, GPX3, GSHB, GSTP1, IDHC, LY6G6E, MASP2, NADC, NAPSA, PA2G4, PARK7, PCBP1, PRDX2, PTD012, QPCT, SBP1 and STIP1 or a transcriptional or a post-translational variant thereof in the test urine sample.

In another embodiment, the first step of the method to assess bladder cancer comprises measuring one or more biomarkers selected from TTHY, CATD H, CATD K, ENOA, FRIL, GSHB, GSTP1, IDCH, MASP2, PRDX2, AMY, APOA1, GSN40, GSN80 and RETBP or a transcriptional or a post-translational variant thereof in the test urine sample.

In another embodiment, the first step of the method to assess bladder cancer comprises measuring one or more biomarkers selected from TTHY, CATD H, CATD K, ENOA, FRIL, GSHB, GSTP1, IDCH, MASP2 and PRDX2 or a transcriptional or a post-translational variant thereof in the test urine sample.

In another embodiment, the first step of the method to assess bladder cancer comprises measuring one or more biomarkers selected from TTHY, AMY, APOA1, GSN40 and GSN80 or a transcriptional or a post-translational variant thereof in the test urine sample.

In another embodiment, the first step of the method to assess bladder cancer comprises measuring APOA1 and RETBP or a transcriptional or a post-translational variant thereof in the test urine sample.

In another embodiment, the first step of the method to assess bladder cancer comprises measuring AMY, APOA1 and GSN40 or a transcriptional or a post-translational variant thereof in the test urine sample.

In another embodiment, the first step of the method to assess bladder cancer comprises measuring AMY, APOA1 and ENOA or a transcriptional or a post-translational variant thereof in the test urine sample.

In another embodiment, the first step of the method to assess bladder cancer comprises measuring APOA1, GSN40 and TTHY or a transcriptional or a post-translational variant thereof in the test urine sample.

In another embodiment, the first step of the method to assess bladder cancer comprises measuring CATD K, GSN80 and IDHC or a transcriptional or a post-translational variant thereof in the test urine sample.

In another embodiment, the first step of the method to assess bladder cancer comprises measuring AMY, APOA1, GSN40 and IDHC or a transcriptional or a post-translational variant thereof in the test urine sample.

In another embodiment, the first step of the method to assess bladder cancer comprises measuring APOA1, GSN40, GSN80 and TTHY or a transcriptional or a post-translational variant thereof in the test urine sample.

In another embodiment, the first step of the method to assess bladder cancer comprises measuring CATD H, ENOA, GSTP1 and PRDX2 or a transcriptional or a post-translational variant thereof in the test urine sample.

In another embodiment, the first step of the method to assess bladder cancer comprises measuring CATD K, ENOA, PRDX2 and TTHY or a transcriptional or a post-translational variant thereof in the test urine sample.

In another embodiment, the first step of the method to assess bladder cancer comprises measuring AMY, CATD K, ENOA, IDHC and PRDX2 or a transcriptional or a post-translational variant thereof in the test urine sample.

In another embodiment, the first step of the method to assess bladder cancer comprises measuring AMY, APOA1, GSN40, GSN80 and TTHY or a transcriptional or a post-translational variant thereof in the test urine sample.

In another embodiment, the first step of the method to assess bladder cancer comprises measuring CATD H, ENOA, GSTP1, MASP2, PRDX2 and TTHY or a transcriptional or a post-translational variant thereof in the test urine sample.

In another embodiment, the first step of the method to assess bladder cancer comprises measuring AMY, APOA1, CATD K, ENOA, IDHC, PRDX2 and TTHY or a transcriptional or a post-translational variant thereof in the test urine sample.

In another embodiment, the first step of the method to assess bladder cancer comprises measuring one or more biomarkers selected from AMY, APOA1, GSN40, GSN80 or a transcriptional or a post-translational variant thereof and one or more biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, OPCT, SBP1, STIP1, TALDO, WDR1 and RETBP or a transcriptional or a post-translational variant thereof in the test urine sample.

In another embodiment, the first step of the method to assess bladder cancer comprises measuring one or more biomarkers selected from AMY, APOA1, GSN40, GSN80 or a transcriptional or a post-translational variant thereof and one or more biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO and WDR1 or a transcriptional or a post-translational variant thereof in the test urine sample.

In another embodiment, the first step of the method to assess bladder cancer comprises measuring one or more biomarkers selected from CATD H, CATD K, ENOA, GSHB, GSTP1, IDHC, PRDX2 and TTHY or a transcriptional or a post-translational variant thereof and one or more biomarkers selected from ACY1, AKR1A1, ALDOB, ANXA4, BIEA, BLVRB, CO3, CPGL1, FRIL, GDIB, GPX3, LMAN2, LY6G6E, MASP2, NADC, NAPSA, PA2G4, PARK7, PCBP1, PDC61, PPAL, PTD012, QPCT, SBP1, STIP1, TALDO, WDR1, AMY, APOA1, GSN40, GSN80 and RETBP or a transcriptional or a post-translational variant thereof.

In another embodiment, the first step of the method to assess bladder cancer comprises measuring one or more biomarkers selected from TTHY, CATD H, CATD K, ENOA, GSTP1, IDHC, MASP2, PRDX2, AMY, APOA1, GSN40, GSN80 and RETBP or a transcriptional or a post-translational variant thereof.

Another embodiment relates to a non-invasive in vitro method that comprises quantifying one or more ratios between two different biomarkers selected from the 40 bladder cancer biomarkers or a transcriptional or a post-translational variant thereof in a test urine sample from an individual and comparing the measurement obtained in the test urine sample to the corresponding standard value in normal urine, wherein variations are indicative of BTCC.

In another embodiment the method allows to determine the progression of the disease when the same protein or proteins is compared from different samples obtained from the same patient at different times within the BTCC evolution.

In another embodiment one or more of the biomarkers of the invention may be used to monitor the efficacy of pharmacological or surgical treatment.

In another embodiment the sample to be analyzed is obtained from an individual not previously diagnosed with BTCC.

In another embodiment the sample to be analyzed is obtained from an individual who has been previously diagnosed with BTCC.

In another embodiment the sample to be analyzed is obtained from an individual currently receiving treatment against BTCC.

In another embodiment the method comprises obtaining an extract of proteins of the sample.

The measuring typically comprises spectrometry or immunoassay. The spectrometry is typically surface enhanced laser desorption/ionization (SELDI) mass spectrometry or matrix assisted laser desorption/ionisation (MALDI) mass spectrometry. Immunoassays include western blot, ELISA (Enzyme-Linked Immunosorbent assay), RIA (Radioimmunoassay), Competitive EIA (Competitive Enzyme Immunoassay), DAS-ELISA (Double Antibody Sandwich-ELISA), immunocytochemical or immunohistochemical techniques, techniques based on the use of biochips or protein microarrays which include specific antibodies, assays based on the precipitation of colloidal gold in formats such as dipsticks; or by affinity chromatography techniques, ligand binding assays or lectin binding assays.

In another embodiment the method for detecting cancer comprises contacting a urine sample with a molecule that specifically binds a bladder cancer biomarker and quantifying this binding.

In another embodiment the detection and quantification of proteins comprises a first step, in which the protein extract of the sample is placed in contact with a composition of one or more specific antibodies against one or more epitopes of the protein or proteins, and a second step, in which the complexes formed by the antibodies and the proteins are quantified.

In another embodiment the specific antibodies used for the detection of proteins are of human origin, humanized or of non-human origin and selected from monoclonal or polyclonal antibodies, intact or recombinant fragments of antibodies, combibodies and Fab or scFv antibody fragments.

Another embodiment relates to the use of one or more peptide sequences derived from the biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO, WDR1, AMY, APOA1, GSN40, GSN80 and RETBP or a transcriptional or a post-translational variant thereof, wherein the biomarkers are present in urine.

Another embodiment of the invention relates to the use of one or more peptide sequences derived from the biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO and WDR1 or a transcriptional or a post-translational variant thereof, wherein the biomarkers are present in urine.

Another embodiment of the invention relates to the use of one or more peptide sequences derived from the cancer biomarkers, wherein variations in the measurement of these biomarkers in a urine test sample in comparison to the corresponding standard value in normal urine are indicative of BTCC.

In another embodiment, at least two biomarkers present in urine or a transcriptional or a post-translational variant thereof are used. In another embodiment, at least three biomarkers present in urine or a transcriptional or a post-translational variant thereof are used. In another embodiment, at least four biomarkers present in urine or a transcriptional or a post-translational variant thereof are used. In another embodiment, at least five biomarkers present in urine or a transcriptional or a post-translational variant thereof are used.

Another embodiment of the invention relates to the use of one or more peptide sequences derived from the biomarkers selected from TTHY, ACY1, AKR1A1, ANXA4, BIEA, BLVRB, CATD H, CATD K, CO3, CPGL1, ENOA, FRIL, GDIB, GPX3, GSHB, GSTP1, IDHC, LY6G6E, MASP2, NADC, NAPSA, PA2G4, PARK7, PCBP1, PRDX2, PTD012, QPCT, SBP1, STIP1, AMY, APOA1, GSN40, GSN80 and RETBP or a transcriptional or a post-translational variant thereof, wherein the biomarkers are present in urine.

Another embodiment of the invention relates to the use of one or more peptide sequences derived from the biomarkers selected from TTHY, ACY1, AKR1A1, ANXA4, BIEA, BLVRB, CATD H, CATD K, CO3, CPGL1, ENOA, FRIL, GDIB, GPX3, GSHB, GSTP1, IDHC, LY6G6E, MASP2, NADC, NAPSA, PA2G4, PARK7, PCBP1, PRDX2, PTD012, QPCT, SBP1 and STIP1 or a transcriptional or a post-translational variant thereof, wherein the biomarkers are present in urine.

Another embodiment of the invention relates to the use of one or more peptide sequences derived from the biomarkers selected from TTHY, CATD H, CATD K, ENOA, FRIL, GSHB, GSTP1, IDCH, MASP2, PRDX2, AMY, APOA1, GSN40, GSN80 and RETBP, or a transcriptional or a post-translational variant thereof, wherein the biomarkers are present in urine.

Another embodiment of the invention relates to the use of one or more peptide sequences derived from the biomarkers selected from TTHY, CATD H, CATD K, ENOA, FRIL, GSHB, GSTP1, IDCH, MASP2 and PRDX2 or a transcriptional or a post-translational variant thereof, wherein the biomarkers are present in urine.

Another embodiment of the invention relates to the use of one or more peptide sequences derived from the biomarkers selected from TTHY, AMY, APOA1, GSN40 and GSN80 or a transcriptional or a post-translational variant thereof, wherein the biomarkers are present in urine.

Another embodiment of the invention relates to the use of the peptide sequences derived from APOA1 and RETBP or a transcriptional or a post-translational variant thereof, wherein the biomarkers are present in urine.

Another embodiment of the invention relates to the use of the peptide sequences derived from AMY, APOA1 and GSN40 or a transcriptional or a post-translational variant thereof, wherein the biomarkers are present in urine.

Another embodiment of the invention relates to the use of the peptide sequences derived from AMY, APOA1 and ENOA or a transcriptional or a post-translational variant thereof, wherein the biomarkers are present in urine.

Another embodiment of the invention relates to the use of the peptide sequences derived from APOA1, GSN40 and TTHY or a transcriptional or a post-translational variant thereof, wherein the biomarkers are present in urine.

Another embodiment of the invention relates to the use of the peptide sequences derived from CATD K, GSN80 and IDHC or a transcriptional or a post-translational variant thereof, wherein the biomarkers are present in urine.

Another embodiment of the invention relates to the use of the peptide sequences derived from APOA1, GSN40, GSN80 and TTHY or a transcriptional or a post-translational variant thereof, wherein the biomarkers are present in urine.

Another embodiment of the invention relates to the use of the peptide sequences derived from AMY, APOA1, GSN40 and IDHC or a transcriptional or a post-translational variant thereof, wherein the biomarkers are present in urine.

Another embodiment of the invention relates to the use of the peptide sequences derived from CATD H, ENOA, GSTP1 and PRDX2 or a transcriptional or a post-translational variant thereof, wherein the biomarkers are present in urine.

Another embodiment of the invention relates to the use of the peptide sequences derived from CATD K, ENOA, PRDX2 and TTHY or a transcriptional or a post-translational variant thereof, wherein the biomarkers are present in urine.

Another embodiment of the invention relates to the use of the peptide sequences derived from AMY, CATD K, ENOA, IDHC and PRDX2 or a transcriptional or a post-translational variant thereof, wherein the biomarkers are present in urine.

Another embodiment of the invention relates to the use of the peptide sequences derived from AMY, APOA1, GSN40, GSN80 and TTHY or a transcriptional or a post-translational variant thereof, wherein the biomarkers are present in urine.

Another embodiment of the invention relates to the use of the peptide sequences derived from CATD H, ENOA, GSTP1, MASP2, PRDX2 and TTHY or a transcriptional or a post-translational variant thereof, wherein the biomarkers are present in urine.

Another embodiment of the invention relates to the use of the peptide sequences derived from AMY, APOA1, CATD K, ENOA, IDHC, PRDX2 and TTHY or a transcriptional or a post-translational variant thereof, wherein the biomarkers are present in urine.

Another embodiment of the invention relates to the use of one or more peptide sequences derived from the biomarkers selected from AMY, APOA1, GSN40 and GSN80 or a transcriptional or a post-translational variant thereof and one or more peptide sequences derived from the biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO, WDR1 and RETBP or a transcriptional or a post-translational variant thereof, wherein the biomarkers are present in urine.

Another embodiment of the invention relates to the use of one or more peptide sequences derived from the biomarkers selected from AMY, APOA1, GSN40 and GSN80 or a transcriptional or a post-translational variant thereof and one or more peptide sequences derived from the biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO and WDR1 or a transcriptional or a post-translational variant thereof, wherein the biomarkers are present in urine.

Another embodiment of the invention relates to the use of one or more peptide sequences derived from the biomarkers selected from CATD H, CATD K, ENOA, GSHB, GSTP1, IDHC, PRDX2 and TTHY or a transcriptional or a post-translational variant thereof and one or more peptide sequences derived from the biomarkers selected from ACY1, AKR1A1, ALDOB, ANXA4, BIEA, BLVRB, CO3, CPGL1, FRIL, GDIB, GPX3, LMAN2, LY6G6E, MASP2, NADC, NAPSA, PA2G4, PARK7, PCBP1, PDC61, PPAL, PTD012, QPCT, SBP1, STIP1, TALDO, WDR1, AMY, APOA1, GSN40, GSN80 and RETBP or a transcriptional or a post-translational variant thereof, wherein the biomarkers are present in urine.

Another embodiment of the invention relates to the use of one or more peptide sequences derived from the biomarkers selected from TTHY, CATD H, CATD K, ENOA, GSTP1, IDHC, MASP2, PRDX2, AMY, APOA1, GSN40, GSN80 and RETBP or a transcriptional or a post-translational variant thereof, wherein the biomarkers are present in urine.

Another embodiment relates to a kit to perform a method as previously defined comprising 1) any combination of antibodies that specifically recognises one or more of these proteins and 2) a carrier in a suitable packaging, the kit being employed to detect the presence of BTCC, to determine the stage or severity of this cancer, to assess the lack of disease after surgical resection, to establish the diagnosis and/or prognosis of this cancer and/or to monitor the effect of the treatment administered to an individual suffering from said cancer.

The kit may comprise a container for housing at least one agent that binds a biomarker present in a urine sample; and instructions for use of the at least one agent for determining status of BTCC. Such kits may comprise a carrier, package or container that is compartmentalized to receive one or more containers such as vials, tubes, and the like, each of the container(s) comprising one of the separate elements to be used in the method. For example, the container(s) can comprise a probe that is or can be labelled and further detected. The probe can be an antibody or polynucleotide specific for a bladder cancer protein or a bladder cancer gene, respectively. Alternatively, the kit can comprise a mass spectrometry (MS) probe. The kit can also include containers containing nucleotide(s) for amplification or silencing of a target nucleic acid sequence, and/or a container comprising a reporter-means, such as a biotin-binding protein, e.g., avidin or streptavidin, bound to a detectable label, e.g., an enzymatic, florescent, or radioisotope label. The kit can include all or part of the amino acid sequence of the bladder cancer biomarkers, or a nucleic acid molecule that encodes such amino acid sequences. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for use, such as those described above. Directions and or other information can also be included on an insert which is included with the kit.

Another embodiment relates to a kit to perform a method as previously defined comprising a biochip.

Another embodiment relates to a kit to perform a method as previously defined comprising a biochip, wherein the biochip comprises antibodies for the detection of one or more biomarkers selected from the 40 bladder cancer biomarkers or a transcriptional or a post-translational variant thereof.

There is a wide range of immunological assays available to detect and/or quantify the formation of specific antigen-antibody complexes; numerous competitive or non-competitive protein-binding assays have been described previously and a large number of these are commercially available. Hence, proteins can be quantified with antibodies such as, for example: monoclonal antibodies, polyclonal antibodies, either intact or recombinant fragments of these, combibodies and Fab or scFv fragments of antibodies, specific for proteins. These antibodies may be of human origin, humanized or of animal origin. On the other hand, they can be labelled or unlabelled and can be used in a wide range of assays. Marker molecules that can be used to label antibodies include radionuclides, enzymes, fluorophores, chemoluminescent reagents, enzymatic substrates or cofactors, enzymatic inhibitors, particles, colorants and derivatives. The higher the antibody binding specificity, the lower the antigen concentration that can be detected.

There are a wide variety of assays well known to those skilled in the art that can be used in the present invention, which use unlabelled antibodies (primary antibodies) and labelled antibodies (secondary antibodies); these techniques include but are not limited to the western blot or western transfer, ELISA (Enzyme-Linked immunosorbent assay), RIA (Radioimmunoassay), Competitive EIA (Competitive enzyme immunoassay), DAS-ELISA (Double antibody sandwich-ELISA), immunocytochemical and immunohistochemical techniques, techniques based on the use of biochips or protein microarrays that include specific antibodies or colloidal precipitation in formats such as dipsticks. Other ways to detect and/or quantify proteins include affinity chromatography techniques, ligand binding assays or lectin binding assays. The preferred embodiments of this aspect of the invention are protein microarrays and double antibody sandwich ELISA (DAS-ELISA). In these immunoassays any antibody, or combination of antibodies, which are specific against one or more epitopes of the 40 proteins of the invention can be used. As an example of one of the many possible formats of this assay, a monoclonal or polyclonal antibody, or a fragment of this antibody, or a combination of these antibodies that recognise one or more epitopes of the 40 proteins of the invention are attached to the surface of a solid phase support and placed in contact with the sample to be analyzed and incubated for a specific time and in appropriate conditions to form the antigen-antibody complexes. After washing in appropriate conditions to eliminate non-specific complexes, an indicator reagent, consisting in a monoclonal or polyclonal antibody, or a fragment of this antibody, or a combination of these and which recognises one or more epitopes of the 40 proteins of the invention, bound to a signal generating molecule, is incubated with the antigen-antibody complexes in appropriate conditions of time and temperature. The presence of one or more proteins selected from the 40 proteins of the invention in the sample to be analyzed is detected and quantified and the signal generated measured. In order to prevent signal variation due to differences in total protein concentration among samples, all measurements are normalized.

As indicated above, the method of the invention involves monitoring the stage of bladder carcinoma by quantitating the differentially-expressed soluble proteins within a urine sample through specific antibodies. As will be appreciated by those skilled in the art, any means for specifically identifying and quantifying these proteins are contemplated.

In the description which follows, the generalized method for obtaining and analyzing the total protein content of human urine samples (herein referred to as the sample) is disclosed. The method involves the processing of the samples, the use of 2D electrophoresis to separate proteins within the sample, the selection of differentially expressed proteins by means of image analysis and statistics of different samples and the use of one or more of the differentially expressed proteins of the invention to generate protein-specific antibodies to be used as bladder cancer markers.

The comparative proteome analysis was performed between samples obtained from healthy individuals (controls) and patients diagnosed with BTCC (Ta, T1-low grade, T1-high grade and T2) in an attempt to identify differentially expressed proteins in the various stages of bladder cancer and during bladder cancer progression. Among all the proteins that showed differential expression, 40 proteins changed more than two-fold reproducibly. They were identified by peptide mass fingerprinting using mass spectrometry and database search.

The present invention will be further illustrated by the following examples. These examples are given by way of illustration only and are not to be construed as limiting.

### I. Identification of Differentially Expressed Proteins

To identify differentially expressed proteins along progression of bladder cancer, protein profiles of healthy urines were compared with those of early-stage and advanced bladder tumor patients using a proteomic approach.

Urine samples (150 in total) were collected from healthy individuals and from patients with transitional bladder cancer visiting the Urology units of Hospitals belonging to the Spanish Public Health Network. These samples were classified as follows:

a) No Carcinoma (63 samples) including:
Controls (CV, 32 samples): patients that had had bladder cancer and had been resected from the tumor and were being controlled (cystoscopy showed no other affectations).
Patients with other urological tract diseases (31 samples, including among others prostate benign hyperplasia, prostate cancer).
   b) BTCC (87 samples): patients diagnosed with the disease at different stages of development including:
Ta (21 samples)
T1-low grade (29 samples)
T1-high grade (19 samples)
T2 (18 samples)
   The BTCC group of samples was accompanied by a biopsy that was the key for their classification in the stages of development of bladder cancer.

### A. Processing of Urine Samples and Separation Of Proteins.

Urine samples were frozen at 80° C. and shipped to the lab in dry ice without breaking the cold chain. Samples were kept at −80° C. until they were processed. The samples were very heterogenic, ranging from light yellow urines to red ones with blood clumps, transparent or containing tissues in suspension. The total volume was also very variable, from 5 to 100 mL. The protein concentration of the samples ranged from 20 μg/mL to 2 mg/mL (150 μg/mL was the average concentration). To, obtain the protein content, samples were thawed on ice and centrifuged at 2000×g for 5 min at 4° C. The supernatant was used to determine protein concentration. The volume necessary to precipitate 100 μg of protein was calculated taking into account that the efficacy of protein precipitation with 15% w/v of trichloroacetic acid (TCA) is 75%. The rest of the urine sample was frozen again at −80° C. and stored for further 2D electrophoresis (if needed). TCA and urine were mixed for 1 hour on ice, and then centrifuged at 16000×g for 20 min at 4° C. to obtain the pellet of precipitated proteins. This pellet was washed with acetone, stored at −20° C. and dried by solvent evaporation.

To perform the two dimension (2D) electrophoresis experiments, the first dimension was IEF (isoelectric focusing), where proteins separate by their charge (pl); the second dimension was SDS-PAGE where proteins separate by their molecular weight. To carry out the first dimension, the dried pellet of proteins was resuspended with 450 μL of rehydratation buffer (Urea 7M, thiourea 2M, CHAPS 2%, IPG buffer 2%, bromophenol blue 0.002%) for 1 hour at room temp. IPG buffer (Amersham, ref#17-600-88) was used so that the IEF ranged from pH 3-10. For the IEF, the Ettan™ IPGphor™ Isoelectric Focusing System from Amersham was used following the manufacturer's directions. The IEF was performed in immobilized pH gradients, named IPG strips, purchased from Amersham (ref#17-6002-45). The solubilized proteins focused in the first dimension in the strips after 16 hours of active rehydratation of the gel at 30 V. Then the voltage was increased to 8000 V, the intensity never being higher than 50 μA per gel. The IEF was finished when the voltage reached 90000 Vhour.

For the second dimension, 26×20 cm 12.5% acrylamide were polymerized in the lab using the Ettan DALT twelve Gel Caster from Amersham. Gels were run in the Ettan DALT twelve Large Format Vertical System and following the manufacturer's instructions until the electrophoresis front left the gel. Gels were silver nitrate dyed using the dye kit from Amersham (17-1150-01) following the manufacturer's directions. Gels were then dried and stored for subsequent image analysis of the protein spots (FIG. 1A). For some urine samples more than one 2D-gel was run.

### B. Analyses of the Protein Spots on the Gels

Every gel was scanned to obtain the map of spots for image analysis. Progenesis PG220 software from Nonlinear Dynamics (UK) was used to analyze image files in a 300 dpi (dot per inch) format and 8 bits/channel. To increase the resolution, the analysis was performed in discrete areas of the gels. For each area, named as A (FIG. 1B), K (FIG. 1C), R (FIG. 1D) and S (FIG. 1E), the best gels were selected and scanned for image analysis. The Progenesis PG220 software transformed the information of the flat image into a 3D image, where the intensity of each spot correlated with its volume and with the relative amount of the corresponding protein in the urine of the individual. With the software tables, the intensities for each spot were obtained in each gel. These raw data were the basis for the subsequent statistical analyses.

To perform the statistical analysis of each individual spot and compare its intensity in two different groups (for instance, CV and Ta), a normal distribution had to be proven in these blocks of data. To compare the spots belonging to two groups, a Student's t test was applied and a p value obtained: this was the theoretical error of assigning a spot to one subgroup or to another one. Only the spots with p values ≦0.05 were selected. The fold change or average ratio of these spots was calculated which either referred to 1) the total spot volume of the sample, that is the theoretical total protein content, or to 2) the constantly expressed protein, the amount of which is constant in every sample, or to 3) a second differentially expressed protein of the same sample.

Also the n value or number of samples was taken into account.

40 proteins showed statistical significance and robustness in their fold change when comparing urine samples from control patients with urine samples from patients diagnosed with transitional bladder cancer.

The identification of those spots was carried out by MALDI-TOF (Matrix-Assisted Laser-Desorption/Ionization Time Of Flight) spectroscopy. Those urine samples whose 2D electrophoresis had shown statistically significant spots were submitted again to 2D electrophoresis and the spots excised from the gel. Proteins were digested and analyzed by MALDI-TOF spectrometer. Peptide mass fingerprint enabled the identification of 40 proteins, that were identified as TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC61, PPAL, PRDX2, PTD012, QPCT, SBP1, STIP1, TALDO, WDR1, AMY, APOA1, GSN40, GSN80 and RETBP (see table 1). Thus, these 40 proteins, that had proven to be differentially expressed in patients diagnosed with BTCC, were identified as biological markers of significant value for the diagnosis, prognosis, monitoring and treatment of the disease.

TABLE 1

| PROTEIN | SEQ ID | SPOT | | | | |
|---|---|---|---|---|---|---|
| SYMBOL | No | No | GI | UniProt | OMIM | PROTEIN NAME |
| TTHY | 1 | S789 | 55669576 | P02766 | 176300 | Transthyretin |
| ACY1 | 2 | K878 | 4501901 | Q03154 | 104620 | Aminoacylase-1 |
| AKR1A1 | 3 | R183 | 1633300 | P14550 | 103830 | Aldehyde reductase |
| ALDOB | 4 | R385 | 113611 | P05062 | 229600 | Aldolase B |
| ANXA4 | 5 | K1273 | 1703319 | P09525 | 106491 | Annexin A4 |
| BIEA | 6 | R211 | 47117734 | P53004 | 109750 | Biliverdin reductase A |
| BLVRB | 7 | S314 | 32891807 | P30043 | 600941 | Flavin reductase |
| | | S322 | | | | |
| CATD H | 8 | S165 | 5822091 | P07339 | 116840 | Cathepsin D |
| CATD K | 9 | K754 | 30582659 | | | |
| | | K760 | | | | |
| | | K780 | | | | |
| CO3 | 10 | A594 | 4557385 | P01024 | 120700 | Complement C3 (fragment) |
| | 11 | A596 | 40786791 | | | |
| CPGL1 | 12 | K629 | 15620780 | Q96KP4 | 169800 | Cytosolic nonspecific |
| | | | | | | dipeptidase |
| ENOA | 13 | R383 | 4503571 | P06733 | 172430 | Alpha enolase |
| | | R058 | | | | |
| | | R054 | | | | |
| | 14 | A519 | 31873302 | | | |
| | | A524 | | | | |
| | | A530 | | | | |
| FRIL | 15 | S444 | 182516 | P02792 | 134790 | Ferritin light chain |
| GDIB | 16 | A499 | 6598323 | P50395 | 600767 | Rab GDP dissociation |
| | | R061 | | | | inhibitor beta |
| GPX3 | 17 | S777 | 404108 | P22352 | 138321 | Plasma glutathione |
| | | | | | | peroxidase |
| GSHB | 18 | K7230 | 8248826 | P48637 | 601002 | Glutathione synthetase |
| GSTP1 | 19 | S346 | 20664359 | P09211 | 134660 | Glutathione S-transferase P |
| IDHC | 20 | R119 | 49168486 | O75874 | 147700 | Cytoplasmic Isocitrate |
| | | | | | | dehydrogenase |
| LMAN2 | 21 | K1187 | 5803023 | Q12907 | None | Vesicular integral- |
| | | K7280 | | | | membrane protein VIP36 |
| | | K7292 | | | | precursor |
| LY6G6E | 22 | K7359 | 55961604 | Q5SRS9 | None | Lymphocyte antigen 6 |
| | | | | | | complex |
| MASP2 | 23 | S775 | 50513646 | O00187 | 605102 | Mannan-binding lectin |
| | | | | | | serine protease 2 |
| NADC | 24 | K1166 | 14603130 | Q15274 | 606248 | Nicotinate-nucleotide |
| | | | | | | pyrophosphorylase |
| NAPSA | 25 | K1213 | 17389633 | O96009 | 605631 | Napsin A |
| | | R276 | | | | |
| PA2G4 | 26 | R056 | 33879698 | Q9UQ80 | 602145 | Proliferation-associated |
| | | | | | | orotein 2G4 |
| PARK7 | 27 | S347 | 50513593 | Q99497 | 602533 | Oncogene DJ1; DJ-1 |
| PCBP1 | 28 | R149 | 5453854 | Q15365 | 601209 | Poly(rC)-binding protein 1 |
| PDC6I | 29 | A627 | 46249756 | Q8WUM4 | 608074 | Programmed cell death 6- |
| | | | | | | interacting protein |
| PPAL | 30 | A641 | 32700070 | P11117 | 171650 | Lysosomal acid |
| | | R072 | | | | phosphatase precursor |
| PRDX2 | 31 | S393 | 1617118 | P32119 | 600538 | Peroxiredoxin 2 |
| PTD012 | 32 | R216 | 48257065 | AAD40375 | None | PTD012 protein |
| QPCT | 33 | K7258 | 525241 | Q16769 | 607065 | Glutaminyl-peptide |
| | | K7346 | | | | cyclotransferase |
| | | K7347 | | | | |
| SBP1 | 34 | A482 | 1374792 | Q13228 | 604188 | Selenium-binding protein 1 |
| STIP1 | 35 | A387 | 54696884 | P31948 | 605063 | Stress-induced- |
| | | | | | | phosphoprotein 1 |
| TALDO | 36 | R205 | 16307182 | P37837 | 602063 | Transaldolase |
| | | R206 | | | | |
| WDR1 | 37 | A372 | 3420181 | O75083 | 604734 | WD-repeat protein 1 |
| AMY | 38 | A638 | 10280622 | P19961 | 104660 | Alpha-amylase |
| | 39 | A636 | 4502085 | P04746 | 104650 | |
| | 40 | A637 | 1633119 | P04745 | 104700 | |
| APOA1 | 41 | S313 | 178775 | P02647 | 107680 | Apolipoprotein A-I |
| | | S319 | | | | |
| | | S782 | | | | |
| GSN40 | 42 | 789 | 17028367 | P06396 | 137350 | Gelsolin |
| | | 2537 | | | | |
| | | 2538 | | | | |
| GSN80 | 43 | A612 | 4504165 | | | |
| RETBP | 44 | S784 | 230284 | P02753 | 180250 | Plasma retinol-binding |
| | | S785 | | | | protein |

As an example, FIG. 2 shows spot R211 corresponding to BIEA in the R area of bidimensional electrophoresis (2D) gel obtained from urine samples of a healthy individual (FIG. 2A), of a patient suffering from cancer at the Ta stage (FIG. 2B), of a patient suffering from cancer at the T1-low grade stage (FIG. 2C), of a patient suffering from cancer at the T1-high grade stage (FIG. 2D) and of a patient suffering from cancer at the T2 stage (FIG. 2E). It can be seen that the intensity of spot R211 is clearly decreased in the samples of the patients with cancer at different stages, when compared to the healthy urine sample. These differences showed to be significant after statistical analysis with Progenesis PG220 software. FIG. 3 shows the intensity of spot R211 in samples CV, Ta, T1-low grade, T1-high grade and T2. The number of samples for each group is indicated in parentheses. FIG. 4 shows the robustness of the intensity measurement for spot R211 in different urine samples. This is expressed as the percentage of the gels that maintain the presence or absence of spot R211 in every gel analyzed. The number of samples for each group is again indicated in parentheses. Table 2 shows the p and fold change values of the statistical analysis for spot R211 in samples of different stages of cancer in comparison to a sample of a non-cancer individual (CV). The fold change of spot R211 was normalized by the total spot volume (TSV) for each individual gel.

TABLE 2

| Type of cancer | p | Fold change |
|---|---|---|
| Ta | 0.0226 | −3.05 |
| T1-low grade | 0.00565 | −2.82 |
| T1-high grade | 0.0212 | −3.84 |
| T2 | 0.0253 | −2.41 |

### II. Antibody Development for the Proteins Found in the Urine Sample

Available polyclonal and/or monoclonal antibodies (either commercially purchased or generated following immunization protocols) were tested for reactivity and sensitivity, the method generally involving preparation of a standard (“dose response”) curve for the protein to be monitored.

### A. Immunization Protocols

### 1. Polyclonal Antibodies

Polyclonal antisera can be raised against a given protein using standard methodologies, such as those disclosed in numerous texts available in the art and known to those generally skilled in the art. In this example, male New Zealand White rabbits are immunized with the protein preparations first in Freund's complete adjuvant (Gibco, Grand Island, N.Y.) and then every month with the protein with incomplete adjuvant for three months. Rabbit sera and sera from mice prior to fusion are used as polyclonal antisera and are shown by standard western blot technique to be reactive with the protein preparations.

### 2. Recombinant Proteins Generation

Proteins found in the gels in too low quantities for immunization are cloned and expressed in E. coli using the pET28b(+) cloning vector. Crude extracts are obtained as described previously (Boronat A., et al., J. Bacteriol. 1981, 147:181-85) and run on a SDS-PAGE gel. Recombinant proteins are excised from the gel and released from acrylamide. For the generation of antibodies against recombinant proteins, the released proteins are directly used to immunize rabbits as described above.

### B. Western Blot Experiments

Protein samples (20 μg of total protein) were mixed with SDS-PAGE gel loading buffer supplemented with 5% β-mercaptoethanol and incubated at 100° C. for 5 min, before being loaded on 6% polyacrylamide gel. Following electrophoresis proteins were transferred to nitrocellulose membranes. Duplicate gels were run and blotted. One membrane was proved with antibodies raised against one or more of the selected 40 proteins of the invention (Santa Cruz Biotech. Inc., Santa Cruz, Calif., USA.) while the second membrane was proved with an antibody raised against actin (Amersham, Little Chalfont, UK) as a control for protein loading. Finally, membranes were hybridised with a secondary antibody conjugated with peroxidase (Amersham) and the chemoluminescent signal was detected using the ECL system (Amersham) with high performance chemiluminescence film (Hyperfilm ECL, Amersham).

### III. Validation of Urine Test Samples Using Western Blot Experiments

Several biomarkers were tested on 40 blinded urine samples comprising:

a) No Carcinoma (12 samples)
b) BTCC including: Ta (8 samples), T1-low grade (6 samples), T1-high grade (6 samples) and T2 (8 samples).

The obtained values of sensitivity, specificity and accuracy are shown in table 3. The values of sensitivity, specificity and accuracy were calculated using these formulas:

$$Accuracy = \frac{( True negatives + True positives )}{Number of samples}$$

$$Sensitivity = \frac{True positives}{( True positives + False negatives )}$$

$$Specificity = \frac{True negatives}{( True negatives + False positives )}$$

For example, the biomarker AMY shows sensitivity of 89% and specificity of 75% for an accuracy value of 85%.

TABLE 3

| | Controls | | Tumors | | | | |
|---|---|---|---|---|---|---|---|
| | True | False | False | True | | | |
| Markers | negatives | negatives | positives | positives | Accuracy | Specificity | Sensitivity |
| AMY | 9 | 3 | 3 | 25 | 85% | 75% | 89% |
| APOA1 | 9 | 2 | 3 | 26 | 88% | 75% | 93% |
| CATD H | 6 | 5 | 6 | 23 | 73% | 50% | 82% |
| CATD K | 8 | 4 | 4 | 24 | 80% | 67% | 86% |
| ENOA | 12 | 13 | 0 | 15 | 68% | 100% | 54% |
| GSHB | 12 | 18 | 0 | 10 | 55% | 100% | 36% |
| GSN40 | 7 | 0 | 5 | 28 | 88% | 58% | 100% |
| GSN80 | 8 | 6 | 4 | 22 | 75% | 67% | 79% |
| GSTP1 | 6 | 3 | 6 | 25 | 78% | 50% | 89% |
| IDHC | 9 | 7 | 3 | 21 | 75% | 75% | 75% |
| PRDX2 | 11 | 11 | 1 | 17 | 70% | 92% | 61% |
| TTHY | 6 | 3 | 6 | 25 | 78% | 50% | 89% |

By a binomial logistic regression of the data obtained in these experiments it was possible to obtain different combinations of biomarkers which generally increased the sensitivity and the specificity of the diagnostic method, as shown in table 4.

TABLE 4

| | Controls | | Tumors | | | | |
|---|---|---|---|---|---|---|---|
| | True | False | False | True | | | |
| Markers | negatives | negatives | positives | positives | Accuracy | Specificity | Sensitivity |
| APOA1, RETBP | 9 | 2 | 3 | 26 | 88% | 75% | 93% |
| AMY, APOA1, GSN40 | 9 | 3 | 3 | 25 | 85% | 75% | 89% |
| AMY, APOA1, ENOA | 9 | 3 | 3 | 25 | 85% | 75% | 89% |
| APOA1, GSN40, | 11 | 1 | 1 | 27 | 95% | 92% | 96% |
| TTHY | | | | | | | |
| CATD K, GSN80, | 7 | 2 | 5 | 26 | 83% | 58% | 93% |
| IDHC | | | | | | | |
| APOA1, GSN40, | 10 | 1 | 2 | 27 | 93% | 83% | 96% |
| GSN80, TTHY | | | | | | | |
| AMY, APOA1, | 9 | 2 | 3 | 26 | 88% | 75% | 93% |
| GSN40, IDHC | | | | | | | |
| CATD H, ENOA, | 9 | 4 | 3 | 24 | 83% | 75% | 86% |
| GSTP1, PRDX2 | | | | | | | |
| CATD K, ENOA, | 8 | 3 | 4 | 25 | 83% | 67% | 89% |
| PRDX2, TTHY | | | | | | | |
| AMY, CATD K, | 7 | 4 | 5 | 24 | 78% | 58% | 86% |
| ENOA, IDHC, PRDX2 | | | | | | | |
| AMY, APOA1, | 11 | 1 | 1 | 27 | 95% | 92% | 96% |
| GSN40, GSN80, | | | | | | | |
| TTHY | | | | | | | |
| CATD H, ENOA, | 10 | 2 | 2 | 26 | 90% | 83% | 93% |
| GSTP1, MASP2, | | | | | | | |
| PRDX2, TTHY | | | | | | | |
| AMY, APOA1, CATD | 10 | 3 | 2 | 25 | 88% | 83% | 89% |
| K, ENOA, IDHC, | | | | | | | |
| PRDX2, TTHY | | | | | | | |

### DESCRIPTION OF THE DRAWINGS

FIG. 1: Bidimensional electrophoresis (2D) gel obtained from a urine sample and showing the four different studied areas A, K, R and S (FIG. 1A), area A (FIG. 1B), area K (FIG. 1C), area R (FIG. 1D) and area S (FIG. 1E).

FIG. 2: Spot R211 corresponding to BIEA in the R area of Bidimensional electrophoresis (2D) gel obtained from urine samples of a healthy individual (FIG. 2A), of a patient suffering from cancer at the Ta stage (FIG. 2B), of a patient suffering from cancer at the T1-low grade stage (T1-LG, FIG. 2C), of a patient suffering from cancer at the T1-high grade stage (T1-HG, FIG. 2D) and of a patient suffering from cancer at the T2 stage (FIG. 2E).

FIG. 3: Intensity of spot R211 in samples CV, Ta, T1-low grade (T1-LG), T1-high grade (T1-HG) and T2. The number of samples for each group is indicated in parentheses.

FIG. 4: Robustness of the intensity measurement for spot R211 in different urine samples. This is expressed as the percentage of the gels that maintain the presence or absence of spot R211 in every gel analyzed. The number of samples for each group is indicated in parentheses.

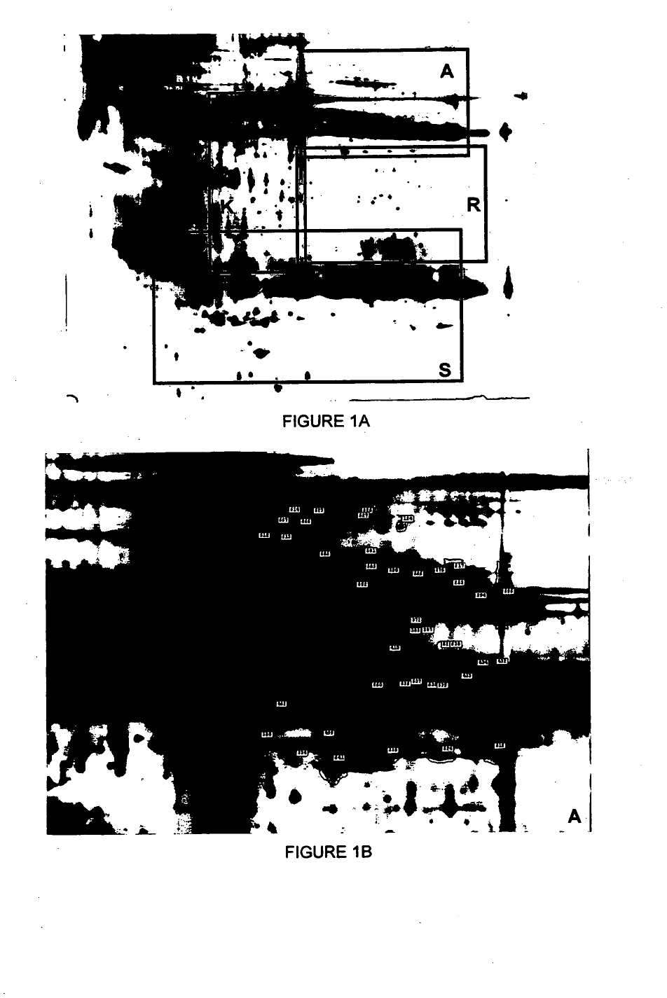

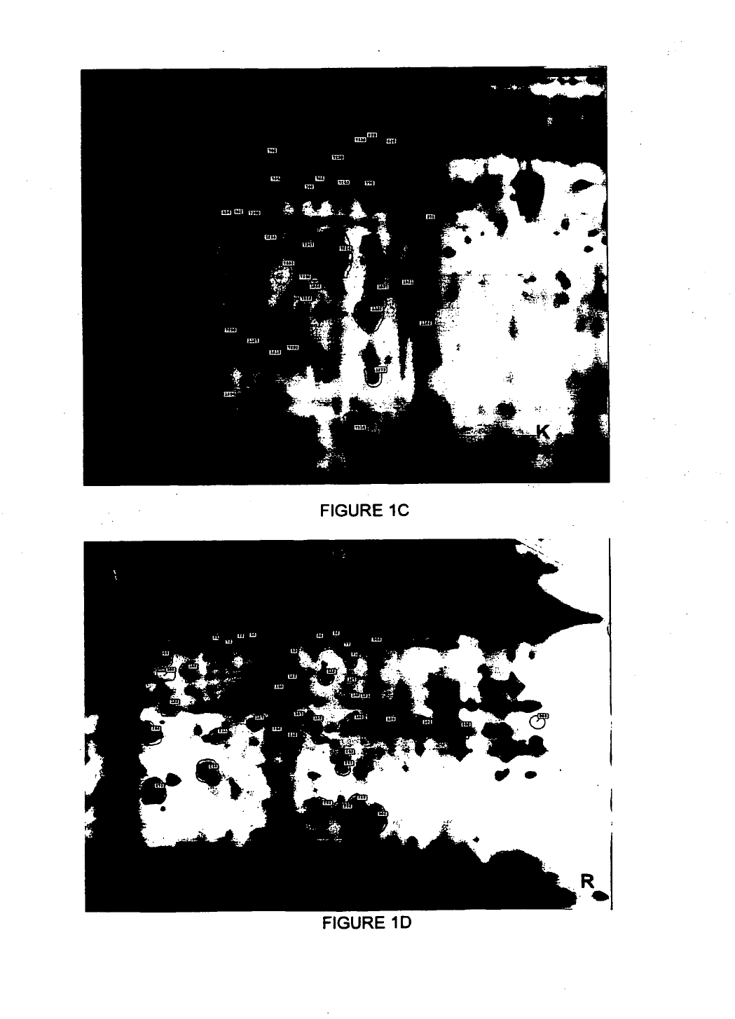

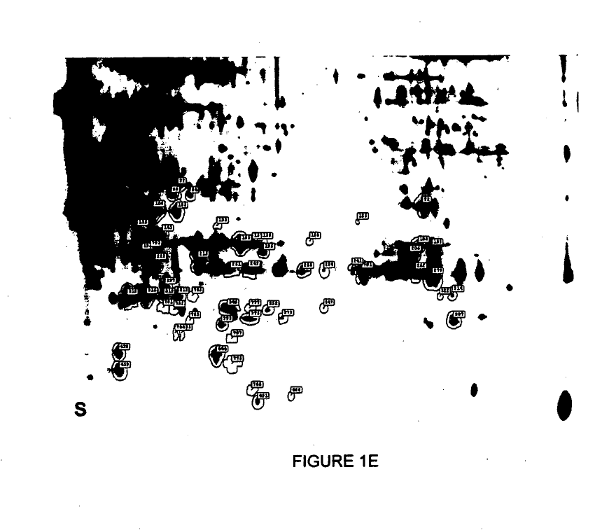

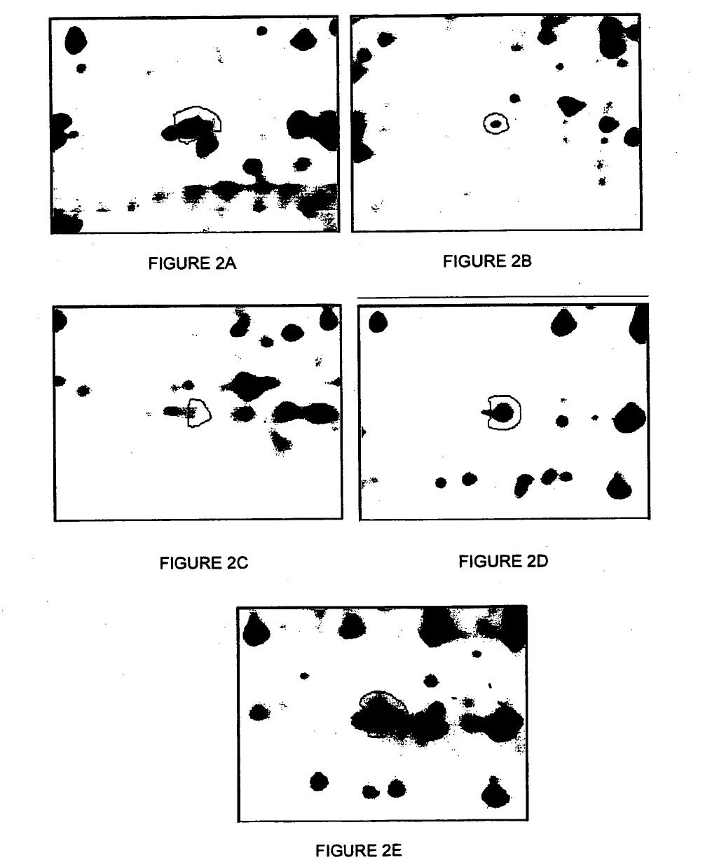

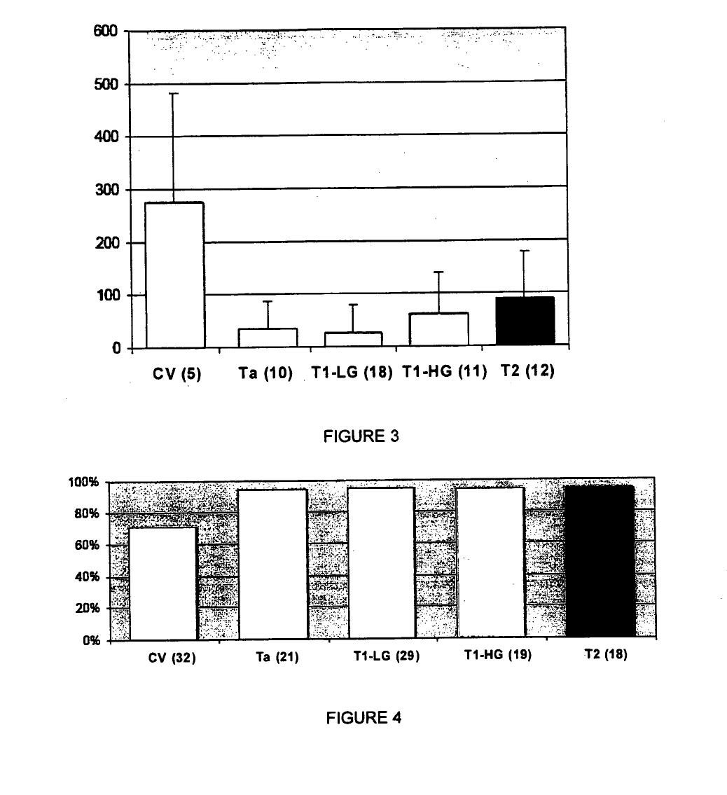

1. A non-invasive in vitro method that comprises:
a) detecting and quantifying at least two biomarkers in a urine test sample from an individual; wherein the at least two biomarkers are selected from the group consisting of TTHY (SEQ ID 1), ACY1 (SEQ ID 2), AKR1A1 (SEQ ID 3), ALDOB (SEQ ID 4), ANXA4 (SEQ ID 5), BIEA (SEQ ID 6). BLVRB (SEQ ID 7), CATD H (SEQ ID 8), CATD K (SEQ ID 9), CO3 (SEQ ID 10, SEQ ID 11), CPGL1 (SEQ ID 12), ENOA (SEQ ID 13, SEQ ID 14), FRIL (SEQ ID 15), GDIB (SEQ ID 16), GPX3 (SEQ ID 17), GSHB (SEQ ID 18), GSTP1 (SEQ ID 19), IDHC (SEQ ID 20). LMAN2 (SEQ ID 21), LY6G6E (SEQ ID 22), MASP2 (SEQ ID 23), NADC (SEQ ID 24), NAPSA (SEQ ID 25), PA2G4 (SEQ ID 26), PARK7 (SEQ ID 27), PCBP1 (SEQ ID 28), PDC6I (SEQ ID 29), PPAL (SEQ ID 30), PRDX2 (SEQ ID 31), PTD012 (SEQ ID 32), QPCT (SEQ ID 33), SBP1 (SEQ ID 34). STIP1 (SEQ ID 35), TALDO (SEQ ID 36), WDR1 (SEQ ID 37), AMY (SEQ ID 38, SEQ ID 39, SEQ ID 40), APOA1 (SEQ ID 41), GSN40 (SEQ ID 42), GSN80 (SEQ ID 43) and RETBP (SEQ ID 44) or a transcriptional or a post-translational variant thereof, and
b) comparing the expression measurement obtained in a) in the urine test sample to the corresponding standard value in normal urine, wherein variations in the measurement obtained in a) compared to the corresponding standard value in normal urine are indicative of transitional cell carcinoma of the bladder.

2. (canceled)

3. Method according to claim 1, wherein the step a) comprises detecting and quantifying at least two biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC6I, PPAL, PRDX2, PTD012, QPCT, SBP1, STIP1, TALDO and WDR1 or a transcriptional or a post-translational variant thereof.

4. Method according to claim 1, wherein the step a) comprises detecting and quantifying at least two biomarkers selected from TTHY, AMY, APOA1, GSN40 and GSN80 or a transcriptional or a post-translational variant thereof.

5. Method according to claim 1, wherein the step a) comprises detecting and quantifying one or more biomarkers selected from AMY, APOA1, GSN40 and GSN80 or a transcriptional or a post-translational variant thereof and one or more biomarkers selected from TTHY, ACY1, AKR1A1, ALDOB, ANXA4, BIEA, BLVRB, CATD H, CATD K, CO3, CPGL1, ENOA, FRIL, GDIB, GPX3, GSHB, GSTP1, IDHC, LMAN2, LY6G6E, MASP2, NADC, NAPSA, PA2G4, PARK7, PCBP1, PDC6I, PPAL, PRDX2, PTD012, QPCT, SBP1, STIP1, TALDO and WDR1 or a transcriptional or a post-translational variant thereof.

6. Method according to claim 1, wherein the step a) comprises detecting and quantifying one or more biomarkers selected from CATD H, CATD K, ENOA, GSHB, GSTP1, IDHC, PRDX2 and TTHY or a transcriptional or a post-translational variant thereof and one or more biomarkers selected from ACY1, AKR1A1, ALDOB, ANXA4, BIEA, BLVRB, CO3, CPGL1, FRIL, GDIB, GPX3, LMAN2, LY6G6E, MASP2, NADC, NAPSA, PA2G4, PARK7, PCBP1, PDC6I, PPAL, PTD012, QPCT, SBP1, STIP1, TALDO, WDR1, AMY, APOA1, GSN40, GSN80 and RETBP or a transcriptional or a post-translational variant thereof.

7. Method according to claim 1, wherein the step a) comprises detecting and quantifying at least two biomarkers selected from TTHY, CATD H, CATD K, ENOA, GSTP1, IDHC, MASP2, PRDX2, AMY, APOA1, GSN40, GSN80 and RETBP or a transcriptional or a post-translational variant thereof.

8. (canceled)

9. Method according to claim 1, wherein the step a) comprises detecting and quantifying at least three biomarkers selected from the group as defined in claim 1.

10. Method according to claim 1, wherein the step a) comprises detecting and quantifying at least four biomarkers selected from the group as defined in claim 1.

11. Method according to claim 1, wherein the step a) comprises detecting and quantifying at least five biomarkers selected from the group as defined in claim 1.

12. Method according to claim 1, wherein the step a) comprises detecting and quantifying the biomarkers APOA1, GSN40 and TTHY or the respective transcriptional or post-translational variants thereof.

13. Method according to claim 1, wherein the step a) comprises detecting and quantifying the biomarkers APOA1, GSN40, GSN80 and TTHY or the respective transcriptional or post-translational variants thereof.

14. Method according to claim 1, wherein the step a) comprises detecting and quantifying the biomarkers AMY, APOA1, GSN40, GSN80 and TTHY or the respective transcriptional or post-translational variants thereof.

15. Method according to claim 1, wherein the step a) comprises detecting and quantifying the biomarkers CATD H, ENOA, GSTP1, MASP2, PRDX2 and TTHY or the respective transcriptional or post-translational variants thereof.

16. Method according to claim 1, wherein the step a) comprises detecting and quantifying the biomarkers AMY, APOA1, CATD K, ENOA, IDHC, PRDX2 and TTHY or the respective transcriptional or post-translational variants thereof.

17. Method according to claim 1 which is employed to detect the presence of transitional cell carcinoma of the bladder, to determine the stage or severity of this cancer, to assess the lack of disease after surgical resection, to establish the diagnosis and/or prognosis of this cancer and/or to monitor the effect of the treatment administered to an individual suffering from the said cancer.

18. Method according to claim 1 in which the urine sample to be analyzed is obtained from an individual not previously diagnosed with transitional cell carcinoma of the bladder.

19. Method according to claim 1 in which the urine sample to be analyzed is obtained from an individual who has been previously diagnosed with transitional cell carcinoma of the bladder.

20. Method according to claim 1 in which the urine sample to be analyzed is obtained from an individual receiving treatment against transitional cell carcinoma of the bladder.

21. Method according to claim 1 characterised in that the detection and quantification of biomarkers comprises a first step, in which the protein extract of the urine sample is placed in contact with a composition of at least two specific antibodies specific to one or more epitopes of the biomarkers of claim 1, and a second step, in which the resulting antibody-protein complexes are quantified.

22. Method according to claim 21 characterised in that the said antibodies are of human origin, humanized or of non-human origin and selected from monoclonal or polyclonal antibodies, intact or recombinant fragments of antibodies, combibodies and Fab or scFv antibody fragments.

23. Method according to claim 21 characterised in that in the detection and quantification of the resulting antibody-protein complexes, the techniques used are selected from the group consisting of: western blot, ELISA (Enzyme-Linked Immunosorbent assay), RIA (Radioimmunoassay), Competitive EIA (Competitive Enzyme Immunoassay), DAS-ELISA (Double Antibody Sandwich-ELISA), immunocytochemical or immunohistochemical techniques, techniques based on the use of biochips or protein microarrays that include specific antibodies, assays based on the precipitation of colloidal gold in formats such as dipsticks; or by affinity chromatography techniques, ligand binding assays or lectin binding assays.

24. Method according to claim 1, wherein the method is used to monitor the efficacy of pharmacological or surgical treatments administered to an individual suffering from transitional cell carcinoma of the bladder.

25. Method according to claim 1, wherein the method is used to determine the progression of the disease when the same at least two biomarkers are compared from different urine samples from the same patient obtained at different times within the evolution of the transitional cell carcinoma of the bladder.

26. Method according to claim 1, wherein the at least two biomarkers are peptide sequences.

27.-42. (canceled)

43. Method according to claim 1, wherein the method is used to screen for, identify, develop and/or evaluate the efficacy of therapeutic agents to treat transitional cell carcinoma of the bladder.

44. A kit to perform a method as defined in claim 1 comprising 1) at least two antibodies which independently specifically recognise one of the biomarkers of claim 1 and 2) a carrier in a suitable packaging.

45. A kit according to claim 44 that is employed to detect the presence of transitional cell carcinoma of the bladder, to determine the stage or severity of this cancer, to assess the lack of disease after surgical resection, to establish the diagnosis and/or prognosis of this cancer and/or to monitor the effect of the treatment administered to an individual suffering from said cancer.

46. A kit according to claims 44 comprising a biochip.

47. A kit according to claim 46, wherein the biochip comprises antibodies for the detection of biomarkers selected from the group as defined in claim 1.
